(19) United States
(12) Patent Application Publication
(10) Pub. No.: US 2025/0375413 A1
(43) Pub. Date: Dec. 11, 2025

(54) METHOD OF INHIBITING LIPASE ACTIVITY IN AGING

(71) Applicant: The Regents of the University of California, Oakland, CA (US)

(72) Inventors: Geert W. Schmid-Schoenbein, Del Mar, CA (US); Frank A. DeLano, San Diego, CA (US)

(21) Appl. No.: 19/233,693

(22) Filed: Jun. 10, 2025

Related U.S. Application Data
(60) Provisional application No. 63/658,240, filed on Jun. 10, 2024.

(51) Int. Cl. A61K 31/365; A61K 31/245; A61K 31/536; A61P 1/00; A61P 1/16; A61P 3/10; A61P 9/12; A61P 11/00; A61P 13/12; A61P 25/14; A61P 25/28

(52) U.S. Cl. CPC A61K 31/365; A61K 31/245; A61K 31/536; A61P 1/00; A61P 1/16; A61P 3/10; A61P 9/12; A61P 11/00; A61P 13/12; A61P 25/14; A61P 25/28

(57) ABSTRACT

Provided herein are methods of general biological aging in a subject, including, but not limited to specific presentations of biological aging such as mild cognitive impairment, dementia, unstable blood pressure, collagen degradation, insulin-receptor cleavage, type 2 diabetes, kidney, lung, or intestine degradation which include administering the subject a therapeutically effective amount of a lipase inhibitor.

### CROSS-REFERENCE TO RELATED APPLICATIONS

This application claims priority to U.S. Provisional Patent Application No. 63/658,240, filed on Jun. 10, 2024. The disclosure of the prior application is considered part of the disclosure of this application, and is incorporated herein by reference in its entirety.

### BACKGROUND

Aging is accompanied by a loss of numerous cell and tissue functions, clinically manifest co-morbidities with increased susceptibility to diseases, and eventually by full organ failure at the time of death. A spectrum of biological processes (e.g., cell and mitochondrial functions, stem cell proliferation and differentiation, genetic lesions, histones, DNA repair mechanisms, epigenetics, protein folding, intra- and inter-cellular signaling, nutrient utilization) become dysregulated, unstable, and exhausted. Vascular and immunological cell functions become impaired with pathological restructuring and development of age-related risk factors and diseases. Different tissues share molecular and cellular mechanisms for micro- and macrovascular pathologies in aging.

Aging is also accompanied by chronic low-grade markers for inflammation. Since the inflammatory cascade fundamentally serves tissue repair, a chronic mechanism exists in aging that causes tissue damage. In all organs, the cells and the extracellular matrix are degrading, for which mechanisms due to reactive oxygen species, radiation exposure, and repeat small injuries have been proposed. However, none has been universally accepted to explain the source of cell dysfunctions and inflammation in aging.

### SUMMARY

Provided herein are methods of reversing accumulation of a lipase in an organ of a subject. The methods include selecting a subject having or at risk of accumulation of a lipase in the organ; and administering a therapeutically effective amount of a lipase inhibitor to the subject, thereby reversing accumulation of the lipase in the organ of the subject.

Also provided herein are methods of reversing cellular damage in an organ of a subject. The methods include selecting a subject having or at risk of cellular damage to the organ; and administering a therapeutically effective amount of a lipase inhibitor, thereby reversing cellular damage in the organ of the subject.

Also provided herein are methods of treating type 2 diabetes. The methods include administering a therapeutically effective amount of a lipase inhibitor to the subject, thereby reducing a cleavage of one or more insulin receptors by a lipase in an organ of the subject.

In some embodiments, the subject is at least 40 years old. In some embodiments, the subject is at least 50 years old. In some embodiments, the subject is at least 60 years old. In some embodiments, the subject is not at risk of developing shock and/or septic shock. In some embodiments, the subject does not have HIV. In some embodiments, the organ is one or more of the small intestine, liver, lung, heart, kidney, brain, or skin. In some embodiments, the organ is the brain. In some embodiments, selecting comprises selecting a subject with a brain disease or condition. In some embodiments, the brain disease or condition is one or more of mild cognitive impairment, Alzheimer's Disease, dementias including frontotemporal dementia, epilepsy or other seizure disorders, mental disorder, multiple sclerosis, Huntington's Disease, Parkinson's Disease, amyotrophic lateral sclerosis, meningitis, encephalitis, brain cancer, Crutzfeldt-Jakob disease, chronic traumatic encephalopathy, long-haul COVID-associated dementia, or stroke.

In some embodiments, the organ is the heart. In some embodiments, selecting a subject comprises selecting a subject with heart disease or a heart condition. In some embodiments, the heart disease or condition is one or more of coronary heart disease, angina, unstable angina, heart failure, cardiac arrhythmias, valve disease, high blood pressure, heart arrhythmias, endocarditis, pericardial disease, or cardiomyopathy. In some embodiments, the organ is the kidney. In some embodiments, selecting a subject at risk comprises selecting a subject with a kidney disease or condition. In some embodiments, the kidney disease or condition is one or more of chronic kidney disease, diabetic kidney disease, acute kidney injury, kidney stones, kidney infections, including pyelonephritis, kidney cysts, or kidney cancer. In some embodiments, is the liver. In some embodiments, selecting a subject comprises selecting a subject with a liver disease or condition. In some embodiments, the liver disease or condition is one or more of hepatitis A, hepatitis B, hepatitis C, autoimmune hepatitis, primary biliary cholangitis, primary sclerosing cholangitis, hemochromatosis, Wilson's disease, alpha-1 antitrypsin deficiency, liver cancer, bile duct cancer, liver adenoma, nonalcoholic fatty liver disease, or nonalcoholic steatohepatitis.

In some embodiments, the lipase inhibitor is a competitive inhibitor. In some embodiments, the lipase inhibitor is one or more of tetrahydrolipstatin (orlistat), cetilistat, valilactone, percyquinin, panclicins A-E, ebelactone A and B, vibralactone, esterastin, nafamostat mesylate (FUT-175), and lipstatin.

In some embodiments, the therapeutically effective amount of the lipase inhibitor is less than about 10% of the subject's digestive enzyme activity. In some embodiments, the therapeutically effective amount of the lipase inhibitor is less than about 10 μM. In some embodiments, the therapeutically effective amount of the lipase inhibitor is less than about 5 μM. In some embodiments, the lipase inhibitor is enterally administered, intraperitoneally administered, intravenously administered, intramuscularly administered, subcutaneously administered, intracutaneously administered, orally administered, intranasally administered, intrapulmonarily administered, intrarectally administered, or administered by a telemetry-controlled external or implanted infusion pump. In some embodiments, the telemetry-controlled infusion pump is directed toward the organ.

In some embodiments, the lipase inhibitor is administered for more than about 1 week. In some embodiments, the lipase inhibitor is administered for more than about 2 weeks. In some embodiments, the lipase inhibitor is administered for more than about 4 weeks. In some embodiments, the lipase inhibitor is administered as a liposome composition or as a nanoparticle encapsulation. In some embodiments, the lipase inhibitor is administered as an eye drop. In some embodiments, the lipase is administered for more than 1 week.

Also provided herein are methods of treating dementia in a subject. The methods include administering a therapeutically effective amount of a lipase inhibitor to the subject, thereby reducing or reversing an accumulation of lipase in a brain of the subject.

Also provided herein are pharmaceutical compositions for the treatment of aging or age-related conditions, the pharmaceutical composition comprising a lipase inhibitor.

In some embodiments, the age-related condition affects an of one or more of the brain, spinal cord, heart, kidney, muscle, liver, or lung. In some embodiments, the age-related condition affects an organ of one or more of the brain, heart, or muscle. In some embodiments, the organ is the brain. In some embodiments, the age-related condition is one or more of mild cognitive impairment, Alzheimer's Disease, dementias including frontotemporal dementia, age-related loss of neuronal function, including but not limited to memory, balance, sensation, pain, epilepsy or other seizure disorders, mental disorder, multiple sclerosis, Huntington's Disease, Parkinson's Disease, amyotrophic lateral sclerosis meningitis, encephalitis, brain cancer, or transient ischemic strokes. In some embodiments, the organ is the heart. In some embodiments, the age-related condition is one or more of coronary heart disease, angina, unstable angina, heart failure, valve disease high blood pressure, heart arrhythmias, endocarditis, pericardial disease, and cardiomyopathy. In some embodiments, the organ is muscle. In some embodiments, the age-related condition is one or more of fibromyalgia, myositis, including polymyositis and dermatomyositis, muscular dystrophy, myasthenia gravis, amyotrophic lateral sclerosis, rhabdomyolysis, cardiomyopathy, sarcopenia, Charcot-Marie-Tooth disease, multiple sclerosis, myopathy, peripheral neuropathy, or spinal muscular atrophy. In some embodiments, the organ is the kidney. In some embodiments, the age-related condition is one or more of acute kidney injury, kidney stones, kidney infections, including pyelonephritis, kidney cysts, the subject being in need of renal dialysis, or kidney cancer. In some embodiments, the organ is the liver. In some embodiments, the age-related condition is one or more of hepatitis A, hepatitis B, hepatitis C, autoimmune hepatitis, primary biliary cholangitis, primary sclerosing cholangitis, hemochromatosis, Wilson's disease, alpha-1 antitrypsin deficiency, liver cancer, bile duct cancer, liver adenoma, nonalcoholic fatty liver disease, or nonalcoholic steatohepatitis.

In some embodiments, the lipase inhibitor is a competitive inhibitor. In some embodiments, the lipase inhibitor is one or more of tetrahydrolipstatin (orlistat), cetilistat, valilactone, percyquinin, panclicins A-E, ebelactone A and B, vibralactone, esterastin, nafamostat mesylate (FUT-175), and lipstatin. In some embodiments, the lipase inhibitor is administered at less than 10% of the subject's digestive enzyme activity. In some embodiments, the lipase inhibitor is less than 10 μM. In some embodiments, the lipase inhibitor is less than 5 μM. In some embodiments, the lipase inhibitor is enterally administered, intraperitoneally administered, intravenously administered, intramuscularly administered, subcutaneously administered, intracutaneously administered, orally administered, intranasally administered, intrapulmonarily administered, intrarectally administered, or administered by a telemetry-controlled external or implanted infusion pump. In some embodiments, the lipase inhibitor is orally administered.

In some embodiments, the lipase inhibitor is administered by a telemetry-controlled infusion pump. In some embodiments, the lipase inhibitor is administered as a liposome composition or a nanoparticle. In some embodiments, the lipase inhibitor is administered as an eye drop. In some embodiments, the telemetry-controlled infusion pump is directed toward the organ. In some embodiments, the lipase inhibitor is administered for more than 1 week. In some embodiments, the lipase inhibitor is administered for more than 2 weeks. In some embodiments, the lipase inhibitor is administered for more than 4 weeks. In some embodiments, any of the pharmaceutical compositions described herein further includes a pharmaceutically acceptable carrier or excipient.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF DRAWINGS

FIGS. 1A-1F. Intestine, liver, and lung in the old, but not the young, were infiltrated by pancreatic trypsin. Pancreatic trypsin label density by immunohistochemistry on tissue cross-sections of young (4 months), old (24 months), and old-treated rats (at age 24 months treated with serine protease inhibitor for 14 days) in (FIGS. 1A-1C) small intestine, liver, and lung. The tissues were labeled with brown substrate except for the liver (with red substrate). The color images for each organ show the IHC labels in the original bright field, and the black/white images depict the IHC label density after digital color extraction. The histograms (FIGS. 1D-1F; right column) show the mean+SD of the trypsin label intensities (digital units). *p<0.05 compared with young group, ‡p<0.05 compared with old untreated rats.

FIGS. 2A-2F. Pancreatic trypsin label density in heart, kidney, and brain is enhanced in the old. Pancreatic trypsin label density by immunohistochemistry on tissue cross-sections of young (4 months), old (24 months), and old-treated rats (at age 24 months treated with serine protease inhibitor for 14 days) in (FIGS. 2A-2C) heart, kidney, and brain. The tissues were labeled with brown substrate except for the liver (with red substrate). The color images for each organ show the IHC labels in the original bright field, and the black/white images depict the IHC label density after digital color extraction. The histograms (FIGS. 2D-2F; right column) show the mean±SD of the trypsin label intensities (digital units). *p<0.05 compared with young group, ‡p<0.05 compared with old untreated rats.

FIGS. 3A-3B. Pancreatic trypsin label density by immunohistochemistry on tissue cross-sections of young (4 months), old (24 months), and old-treated rats (at age 24 months treated with serine protease inhibitor for 14 days) in (FIG. 3A) abdominal skin and (FIG. 3B) in enface view of the mesentery (arterioles (A), venules (V), and capillaries (C)). The tissues were labeled with brown substrate except for the liver (with red substrate). The color images for each organ show the IHC labels in the original bright field, and the black/white images depict the IHC label density after digital color extraction. The histogram (right column) show the mean±SD of the trypsin label intensities (digital units). *p<0.05 compared with young group, ‡p<0.05 compared with old untreated rats.

FIG. 4. Infiltration of pancreatic digestive elastase into organs of the old but not the young. Immunohistochemical detection of pancreatic elastase in young (4 months) and old (24 months) vital tissues. Sections were labeled with brown substrate. The inserts in show control brightfield images without the use of the primary antibody against the digestive enzymes. The histograms (right column) show mean±SD of the image intensity (in digital units after black and white conversion; not shown). The digital measurements were carried out on single larger tissue sections (˜ 4 mm×5 mm) by the placement of a digital window (20 μm×30 μm) with 30 random measurements per section. *p<0.05 compared with the young group. Note the relatively small standard deviation for the label intensities, indicating that each tissue on a length scale of >20 μm was relatively uniformly infiltrated by digestive enzymes. The tissues exhibited non-uniform label intensities on a smaller scale (selected locations marked by arrows).

FIG. 5. Pancreatic lipase accumulates in the organs of the old. Immunohistochemical detection of pancreatic lipase in young (4 months) and old (24 months) vital tissues. Sections were labeled with brown substrate. The inserts in show control brightfield images without the use of the primary antibody against the digestive enzymes. The histograms (right column) show mean±SD of the image intensity (in digital units after black and white conversion; not shown). The digital measurements were carried out on single larger tissue sections (˜ 4 mm×5 mm) by the placement of a digital window (20 μm×30 μm) with 30 random measurements per section. *p<0.05 compared with the young group. Note the relatively small standard deviation for the label intensities, indicating that each tissue on a length scale of >20 μm was relatively uniformly infiltrated by digestive enzymes. The tissues exhibited non-uniform label intensities on a smaller scale (selected locations marked by arrows).

FIG. 6. Pancreatic amylase accumulates in the organs of the old. Immunohistochemical detection of pancreatic amylase in young (4 months) and old (24 months) vital tissues. Sections were labeled with brown substrate. The inserts in show control brightfield images without the use of the primary antibody against the digestive enzymes. The histograms (right column) show mean±SD of the image intensity (in digital units after black and white conversion; not shown). The digital measurements were carried out on single larger tissue sections (˜ 4 mm×5 mm) by the placement of a digital window (20 μm×30 μm) with 30 random measurements per section. *p<0.05 compared with the young group. Note the relatively small standard deviation for the label intensities, indicating that each tissue on a length scale of >20 μm was relatively uniformly infiltrated by digestive enzymes. The tissues exhibited non-uniform label intensities on a smaller scale (selected locations marked by arrows).

FIG. 7. Mucin layer density in the small intestine was reduced in the aged. Enface view of the small intestine (jejunum) in young, old, and old-treated rats (same as in FIG. 1) after labeling mucin with alcian blue. Upper panel: optical focus on the villi tips; lower panel: focus on the crest region between villi. Histograms (right column) show mucin label intensities (mean±SD) at the villi tip and the submucosa.

FIG. 8. Escape of digestive enzymes across the mucosal barrier in the old with reduced mucin layer. Enface view of small intestine with dual labeling of mucin (blue) and pancreatic trypsin and amylase by immunohistochemistry (brown). Histograms of the enzyme label density were measured by optical intensity on images after digital color extraction of the brown enzyme labels (shown in black and white panels). Trypsin and amylase measurements were carried out separately on the villi (yellow windows) and between villi at the submucosa level (red windows). *p<0.05 compared with young group, ‡p<0.05 compared with old untreated rats.

FIGS. 9A-9B. Trypsin leaked at the tip of the intestinal villi. (FIG. 9A) Cross-section (30 μm thickness) of the small intestine villi from young and old rats with dual staining for mucin (blue) and trypsin (brown). The ubiquitous globular mucin and the mucin attached to the villi tips (thick arrows) in young animals was reduced in the old (thin arrows), accompanied by the entry of trypsin into the villus interstitial space. (FIG. 9B) Thin cross-section (1 μm) of intestinal villus of old rat, dual labeled with mucin and trypsin, and shown separately after digital color extraction (middle and right panel). Sites of entry of trypsin (arrows, middle panel) were in goblet cells with depleted mucin (right panel). Traces of trypsin immunolabel were present in the lymphangion (L), and the microvasculature (M) as well as prominently in the intestinal serosa(S).

FIG. 10. Collagen damage in the old intestine, liver, and lung was higher than in the young and attenuated by the blockade of leaking digestive enzymes. Hybridizing peptides revealed tissue sites with collagen damage in the old that was significantly elevated compared to the young. Measurements by digital color extraction of the red peptide label shown in the black/white panels and histograms of digital intensity measurements. In the young, the intestinal villi and the liver had collagen damage higher than in the lung, heart, kidney, and brain. *p<0.05 compared with the young group, ‡p<0.05 compared with old untreated rats.

FIG. 11. Collagen damage in old heart, kidney, and brain was higher than in the young and attenuated by the blockade of leaking digestive enzymes. Hybridizing peptides revealed tissue sites with collagen damage in the old that was significantly elevated compared to the young. Measurements by digital color extraction of the red peptide label shown in the black/white panels and histograms of digital intensity measurements. In the young, the intestinal villi and the liver had collagen damage higher than in the lung, heart, kidney, and brain. *p<0.05 compared with the young group, ‡p<0.05 compared with old untreated rats.

FIG. 12. Collagen damage in the skin of the old is higher than in the young and attenuated by the blockade of leaking digestive enzymes. Hybridizing peptides revealed tissue sites with collagen damage in the old that was significantly elevated compared to the young. Measurements by digital color extraction of the red peptide label shown in the black/white panels and histograms of digital intensity measurements. *p<0.05 compared with the young group, ‡p<0.05 compared with old untreated rats.

FIGS. 13A-13B. Insulin receptor cleavage and hyperglycemia in the old. (FIG. 13A) Immunolabel density of the extracellular domain of the insulin receptor (brown substrate) in sections of the brain cortex of young and old rats without and with temporary trypsin treatment. The top row shows original color images and the bottom row insulin receptor density after digital color extraction of the brown substrate with a histogram of the label density measurements. (FIG. 13B) Blood glucose values in the same groups. *p<0.05 compared with young group, ‡p<0.05 compared with old untreated rats.

FIG. 14. Brightfield images of typical capillary networks in villi of the upper jejunum in young and old rats. The capillaries are filled with carbon solution.

### DETAILED DESCRIPTION

The present disclosure describes methods of inhibiting a lipase and decreasing the activity of the lipase outside a gastrointestinal (GI) tract in a subject.

Various non-limiting aspects of these methods are described herein, and can be used in any combination without limitation. Additional aspects of various components of the methods described herein are known in the art.

It must be noted that, as used in the specification and the appended claims, the singular forms “a,” “an” and “the” include plural referents unless the context clearly dictates otherwise.

As used herein, the term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that are within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

As used herein, a “cell” can refer to either a prokaryotic or eukaryotic cell, optionally obtained from a subject or a commercially available source.

### Aging Process

As used herein, the term “aging” refers to the process associated with becoming older. While the term refers especially to human beings, many animals, and fungi, in the broader sense, aging can also refer to single cells within an organism which have ceased dividing (cellular senescence), show reduced cell functions (response to for example growth hormones, insulin) and gene expression. In humans, aging represents the accumulation of changes over time, encompassing physical and psychological changes. For example, aging is accompanied by a loss of cell and tissue functions, clinically manifesting co-morbidities with increased susceptibility to diseases, and eventual by full organ failure. A spectrum of biological processes (e.g., cell and mitochondrial functions, stem cell proliferation and differentiation, genetic lesions, histones, DNA repair mechanisms, epigenetics, protein folding, intra- and inter-cellular signaling, and nutrient utilization) become dysregulated, unstable, and exhausted. Pathophysiological mechanisms in aging can include impaired resistance to molecular stressors, chronic low-grade inflammation, genomic instability, telomere attrition and cellular senescence, epigenetic alterations, loss of protein homeostasis (proteostasis), deregulated nutrient sensing, stem cell exhaustion, and/or altered intercellular communication. Vascular and immunological cell functions become impaired with pathological restructuring and development of age-related risk factors and diseases, while different tissues share molecular and cellular mechanisms for micro- and macrovascular pathologies in aging. Aging is also accompanied by chronic low-grade inflammation, and since the inflammatory cascade fundamentally serves tissue repair, a chronic mechanism can exist in aging that causes tissue damage. In all organs, the cells and the extracellular matrix are known to degrade, for which mechanisms have been proposed to be due to reactive oxygen species, radiation exposure, and repeat small injuries.

Aging is among the greatest known risk factors for most human diseases: of the roughly 150,000 people who die each day across the globe, about two thirds die from age-related causes. Aging is associated with changes in dynamic biological, physiological, environmental, psychological, behavioral, and social processes. As used herein, “symptoms of biological aging” can refer to common signs and symptoms of aging that can include, but are not limited to, degradation of the extracellular matrix, increased susceptibility to infection, greater risk of heat stroke or hypothermia, skin thinning and wrinkling, bones break more easily, joint changes, ranging from minor stiffness to severe arthritis, slowed and limited movement, decrease in overall energy, constipation, urinary incontinence, cognitive impairment (e.g., slowing of thought, memory, and thinking), reduced reflexes and coordination, difficulty with balance, decrease in visual acuity, diminished peripheral vision, hearing loss, whitening or graying of hair, loss of smell, and weight loss in part due to loss of muscle tissue.

Serine protease activity, including serine hydrolase and lipase activity, in organs outside the GI tract has been discovered to serve as a mechanism for chronic and gradual loss of cell and organ functions during aging (e.g., “Autodigestion”). After synthesis in the pancreas, digestive enzymes can be discharged into the small intestine where they degrade large masses of biomolecules. In the small intestine, digestive enzymes are concentrated (e.g., at sub-mM level), fully activated and relatively non-specific to facilitate breakdown of diverse polymeric food sources into lower molecular weight monomeric nutrients. Furthermore, autodigestion of one's own intestine is primarily prevented by compartmentalization of the digestive enzymes in the lumen of the intestine by the mucin/epithelial barrier, and while this barrier is always permeable to small molecular nutrients (e.g., ions, amino acids, or monosaccharides) it generally has a low permeability to larger molecules, such as pancreatic serine proteases. However, sometimes the mucin/epithelial barrier is compromised due to disease or conditions, and sometimes the mucin/epithelial barrier becomes compromised during aging, as older individuals tend to have weaker mucin/epithelial barriers than young individuals.

The present disclosure provides mechanisms for aging due to autodigestion involving serine proteases, including lipase. The methods of the disclosure block serine proteases (e.g., lipase) outside the gastrointestinal tract (GI) tract with minimal effect on serine protease (e.g., lipase) activity inside the GI tract to ameliorate symptoms and diseases of aging due to autodigestion.

### Serine Proteases/Serine Protease Inhibitors

Serine proteases are sometimes referred to as serine endopeptidases, which are enzymes that can cleave peptide bonds in proteins. There are two main categories of serine proteases based on their structure, chymotrypsin-like (trypsin-like) and subtilisin-like. Subtilisin-like serine proteases can be found in prokaryotes and share the same catalytic mechanism as the trypsin-like serine proteases. The chymotrypsin-like/trypsin-like serine proteases contain two beta-barrel domains that converge at a catalytic site. Serine proteases are folded in such a way that they utilize a catalytic triad located in the active site of the enzyme, which consists of three amino acids, Histidine 57, Serine 195, and Aspartic acid 102. Additionally, elastase is a serine protease produced by the pancreas that catalyzes cleavage of carboxyl groups present on small hydrophobic amino acids, such as glycine, alanine, and valine. The primary role of elastase is the breakdown of elastin, a protein that imparts elasticity to connective tissue.

Serine proteases can be inhibited by serine protease inhibitors, which can include chemical inhibitors as well as proteinaceous inhibitors. In non-limiting embodiments, small molecular weight inhibitors can pass out of the small intestine and into blood, plasma, or other tissues. Sometimes serine protease inhibitors are called SERPINs. Serine protease inhibitors can include competitive inhibitors, non-competitive inhibitors, permeant inhibitors, reversible inhibitors, and irreversible inhibitors. Sometimes serine protease inhibitors block a serine protease by changing the conformational shape of the serine protease, disrupting the active site of the serine protease. Sometimes serine protease inhibitors bind to and block the active site of a serine protease.

Non-limiting examples of serine protease inhibitors include Lepirudin, Bivalirudin, Argatroban, Chymostatin, Benzamidine, Ximelagatran, Rivaroxaban, Idraparinux, Apixaban, Otamixaban, Aprotinin, Dabigatran etexilate, Edoxaban, Letaxaban, Ulinastatin, Darexaban, Nafamostat, Gabexate, Sivelestat, Melagatran, Cholesterol sulfate, Dabigatran, Fondaparinux, Desirudin, Betrixaban, CGS-27023, GW-813893, Berotralstat, Evolocumab, Conestat alfa, Rosmarinic acid, Alpha-1 antitrypsin, Alpha-2 antiplasmin, BIA 10-2472, C1-inhibitor, Camostat, Cospin, CU-2010, CU-2020, Kallistatin, Kazal domain, Maspin, Methoxy arachidonyl fluorophosphonte, Microviridin, Plasminogen activator inhibitor-1, Plasminogen activator inhibitor-2, PMSF, Protein C inhibitor, Protein Z-related protease inhibitor, SERPINA9, SERPINB1, SERPINB3, SERPINB4, SERPINB6, SERPINB7, SERPINB8, SERPINB9, SERPINB13, SERPINE2, SPINTI, Spaostat, and Uterine Serpin.

### Serine Hydrolases/Serine Hydrolase Inhibitors

Serine hydrolases are a broad and diverse family of enzymes that share a common catalytic mechanism involving a serine residue in their active site. These enzymes catalyze the hydrolysis of various chemical bonds-such as esters, amides, and thioesters—by using the hydroxyl group of serine as a nucleophile to attack the substrate. This reaction is typically facilitated by a catalytic triad composed of serine, histidine, and aspartate (or glutamate), which work together to stabilize the transition state and enhance the enzyme's reactivity. Structurally, many serine hydrolases adopt an α/β-hydrolase fold, a common protein architecture that supports their catalytic function. However, some members of this family may have different structural motifs while still employing the same core mechanism. Serine hydrolases are functionally diverse and include several important enzyme classes such as serine proteases (which break down proteins), lipases (which digest fats), esterases, amidases, and thioesterases. These enzymes play essential roles in numerous biological processes, including digestion, neurotransmission, metabolism, immune response, and cell signaling. Their widespread presence and functional versatility make them critical to both normal physiology and the pathology of various diseases.

Lipase is a serine hydrolase that belongs to the α/β-hydrolase fold family, which is structurally distinct from both the trypsin-like and subtilisin-like serine protease families. Lipase is a type of enzyme that plays a crucial role in the digestion and metabolism of dietary fats. Found in various tissues and secretions—including the pancreas, stomach, and intestines—lipases catalyze the hydrolysis of triglycerides into free fatty acids and glycerol. This reaction is essential for the absorption of fats in the small intestine, where bile salts emulsify fat droplets, increasing their surface area and allowing lipases to act more efficiently. Structurally, most lipases belong to the serine hydrolase family and share a common α/β-hydrolase fold, a structural motif that supports their catalytic activity. The active site typically contains a catalytic triad composed of serine, histidine, and aspartic acid residues. These enzymes are unique in that they are often activated only at the oil-water interface, which is where fat digestion occurs in the gut. Human pancreatic lipase, for example, specifically targets the ester bonds at the sn-1 and sn-3 positions of triglycerides, producing two free fatty acids and a monoglyceride. Lipases are not only vital for digestion but also participate in broader physiological processes such as lipid transport, energy storage, and cellular signaling. Their activity is tightly regulated, and imbalances can contribute to metabolic disorders, including obesity and pancreatitis.

Serine hydrolase inhibitors are compounds that block the activity of enzymes within the serine hydrolase family, which includes lipases, esterases, amidases, and serine proteases. These enzymes share a common catalytic mechanism involving a serine residue that acts as a nucleophile to cleave ester, amide, or thioester bonds. Inhibitors typically work by covalently modifying the active site serine, thereby preventing the enzyme from interacting with its natural substrate. Some inhibitors are broad-spectrum, targeting many serine hydrolases, while others are designed to be highly selective, binding only to specific enzymes based on structural or functional differences. One of the most widely used tools in this area is the fluorophosphonate-based activity-based probe (ABP), which irreversibly binds to the active site serine and has been instrumental in profiling enzyme activity and screening for new inhibitors.

In some embodiments, the lipase inhibitor of the methods of the disclosure includes tetrahydrolipstatin (orlistat), cetilistat, valilactone, percyquinin, panclicins A-E, ebelactone A and B, vibralactone, esterastin, diisopropyl fluorophsphate (DFP), nafamostat mesylate (FUT-175 or Futhan), and lipstatin. In some embodiments, the lipase inhibitor can include a derivative of any one of the lipase inhibitors described herein. In some embodiments, the lipase inhibitor can be a serine protease inhibitor that also has inhibitory activity. In some embodiments, the serine protease inhibitor that also has inhibitory activity is nafamostat mesylate (FUT-175 or Futhan).

In some embodiments, the lipase inhibitor of the methods of the disclosure includes one or more natural compounds. In some embodiments, these one or more natural compounds include one or more of resveratrol (Plumbago zeylanica), cocoa, Mentha viridis, mangosteen (Garcinia), white birch, Monascus pigment, kanzinoki, ural licorice, Nepeta japonica Maximowicz, araucaria, Origanum vulgare, alginate bread, houttuynia, tortoiseshell (sponge), horned squash, Salacia reticulata, Luo Zizi (legume), Pu′er tea, brick tea, Kuding tea, black tea, Chrysanthemum morifolium, rose, mushroom, turmeric, dried ginger powder, adzuki bean, buckwheat, apple pomace, green pepper, lotus leaf, grape seed, ginseng or American ginseng, platycladus, chickpea, brown algae, or Hebridean brown algae. In some embodiments, these one or more natural compounds include one or more of curcuminoids; apple polyphenols; green tea catechins such as epigallocatechin gallate (EGCG); saponins, present in legumes and herbs like Camellia sinensis and Panax ginseng; flavonoids like quercetin and kaempferol; tannins; or algae-derived compounds from marine sources. In some embodiments, the one or more natural compounds are administered orally to a subject or a patient.

### Pharmaceutical Compositions for the Treatment of Age-Related Conditions

Any of the methods described herein include the use of pharmaceutical compositions comprising one or more of lipase inhibitors as an active ingredient.

As used herein, the term “pharmaceutical composition” refers to a composition in which an active agent is formulated together with one or more pharmaceutically acceptable carriers. In some embodiments, the composition is suitable for administration to a human or animal subject. In some embodiments, the active agent is present in unit dose amount appropriate for administration in a therapeutic regimen that shows a statistically significant probability of achieving a predetermined therapeutic effect when administered to a relevant population.

Pharmaceutical compositions are typically formulated to be compatible with its intended route of administration. Examples of routes of administration include parenteral, e.g., intravenous, subcutaneous, oral (e.g., capsules or inhalation), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798.

Systemic administration of a pharmaceutical composition as described herein can also be by transmucosal means. For transmucosal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories.

The pharmaceutical compositions can also be prepared in the form of suppositories (e.g., with conventional suppository bases such as cocoa butter and other glycerides) or retention enemas for rectal delivery.

In one embodiment, the pharmaceutical compositions are prepared with carriers that will protect the pharmaceutical compositions against rapid elimination from the body, such as a controlled release formulation, including implants and microencapsulated delivery systems. In some embodiments, the pharmaceutical compositions include a serine protease inhibitor that is linked, conjugated, or fused to another molecule. In some embodiments, the other molecule changes a property of the pharmaceutical composition. In some embodiments, pharmaceutical compositions can be delivered by using nanoparticle encapsulation. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

In some embodiments, the pharmaceutical compositions further include a pharmaceutically acceptable carrier or excipient. For example, the pharmaceutically acceptable carrier may be a liquid or solid filler, diluent, excipient, solvent, or encapsulating material, or a combination thereof. Each component of the carrier must be “pharmaceutically acceptable” in that it must be compatible with the other ingredients of the formulation and is compatible with administration to a subject, for example a human. It must also be suitable for use in contact with any tissues or organs with which it may come in contact, meaning that it must not carry a risk of toxicity, irritation, allergic response, immunogenicity, or any other complication that excessively outweighs its therapeutic benefits. Examples of pharmaceutically acceptable carriers include, but are not limited to, a solvent or dispersing medium containing, for example, water, pH buffered solutions (e.g., phosphate buffered saline (PBS), HEPES, TES, MOPS, etc.), isotonic saline, Ringer's solution, polyol (for example, glycerol, propylene glycol, liquid polyethylene glycol, and the like), alginic acid, ethyl alcohol, and suitable mixtures thereof. In some embodiments, the pharmaceutically acceptable carrier can be a pH-buffered solution (e.g. PBS).

### Methods of Decreasing Lipase Activity Outside of the Gastrointestinal (GI) Tract

Aging and/or age-related diseases or conditions can cause an increase in the permeability of the intestinal barrier to digestive serine proteases and serine hydrolases (e.g., lipase) such that serine protease and serine hydrolase activity may be detectable in the circulation of the subject. Digestive enzymes (e.g., lipase) can leak across the mucin-epithelial barrier into tissues and organs outside the pancreas and intestines where they may damage the extracellular matrix and cell membranes. In some embodiments, damage may include ectodomain receptor cleavage.

The digestive enzymes (e.g., lipase) can cause multiple forms of tissue damage, including cleavage of membrane receptors (e.g. the insulin receptor, growth hormone receptor) and degradation of collagen in organs of the subject. Pancreatic trypsin can also activate prohormones and interfere with physiological signaling due to its ability to cleave a broad spectrum of humoral mediators as well as their receptors. Treatment with administration of a digestive enzyme inhibitor (e.g., serine protease inhibitor, e.g., trypsin inhibitor) can attenuate breakdown of the mucin barrier, reduce the accumulation of digestive enzymes in peripheral organs, as well as cleavage of collagen. For example, interventions against pancreatic trypsin outside the small intestine not only block activation of secondary proteases, but also maintain a spectrum of cell functions (including, but not limited to, immune responses, mitochondrial functions, stem cell proliferation and differentiation, DNA repair mechanisms, epigenetics, protein folding, intra- and inter-cellular signaling, and nutrient utilization).

The compositions described herein can be administered to a subject to treat or prevent diseases, disorders, or conditions described herein. In some embodiments, the present disclosure describes methods of reversing accumulation of a serine hydrolase in an organ of a subject, reversing cellular damage in an organ of a subject, and/or preserving extracellular matrix in an organ of a subject, by selecting a subject at risk of damage to the organ, and administering a therapeutically effective amount of a serine hydrolase inhibitor.

In some embodiments, the present disclosure describes methods of reversing accumulation of a lipase in an organ of a subject, reversing cellular damage in an organ of a subject, and/or preserving extracellular matrix in an organ of a subject, by selecting a subject at risk of damage to the organ, and administering a therapeutically effective amount of a lipase inhibitor.

In some embodiments, the present disclosure describes methods of treating type 2 diabetes by administering a therapeutically effective amount of a serine hydrolase inhibitor (e.g., a lipase inhibitor) to the subject, thereby reducing a cleavage of one or more insulin receptors by a serine hydrolase (e.g., a lipase) in an organ of the subject. As described in Examples 5 and 6, digestive proteases can be involved in the cleavage of insulin receptors. Cleavage of the insulin receptor can be a contributing factor to the development of type 2 diabetes by impairing insulin signaling. Under normal conditions, insulin binds to its receptor on the cell surface, triggering a cascade of intracellular events that promote glucose uptake and regulate metabolism. However, a cleavage process can remove the extracellular domain of the receptor, generating a soluble insulin receptor fragment that is released into the bloodstream. As a result, fewer functional insulin receptors remain on the cell surface, reducing the cell's ability to respond to insulin. This leads to insulin resistance, a hallmark of type 2 diabetes, where cells fail to efficiently take up glucose despite the presence of insulin.

In some embodiments, the methods described herein treat, prevent, or mitigate type two diabetes by reducing the number of insulin receptors cleaved by digestive enzymes (e.g., a lipase or a serine protease) via administration of a lipase inhibitor or a serine protease inhibitor. In some embodiments, the methods disclosed herein increase the number of functional insulin receptors on the cell surface, thereby increasing the cell's ability to respond to insulin. In some embodiments, the lipase inhibitors or serine protease inhibitors of the disclosure reduce the number of insulin receptors cleaved by digestive enzymes in cells found in one or more of the liver, muscle (skeletal and cardiac), adipose tissue, brain, kidney, ovary and testis, pancreas, placenta, intestine, lung, spleen, thymus, or brain (e.g., choroid plexus). In some embodiments, the lipase inhibitors or serine protease inhibitors of the disclosure reduce the number of insulin receptors cleaved by digestive enzymes in cells found in one or more of the liver, adipose tissue, skeletal muscle, or the brain.

In some embodiments, the present disclosure describes methods of treating dementia in a subject by administering a therapeutically effective amount of a serine hydrolase inhibitor (e.g., a lipase inhibitor) to the subject, thereby reducing or reversing an accumulation of a serine hydrolase (e.g., a lipase) in a brain of the subject.

In some embodiments, the present disclosure describes methods of treating a disease or condition of the brain, spinal cord, heart, muscle, kidney, liver, or lung.

In some embodiments, the present disclosure describes methods of decreasing serine hydrolase (e.g., lipase) activity outside a gastrointestinal (GI) tract of a subject. In some embodiments, the methods can inhibit or reduce activity of a serine hydrolase (e.g., lipase) outside a gastrointestinal (GI) tract of a subject, or reduce symptoms of biological aging in a subject. In some embodiments, the methods include administering to a subject in need thereof a therapeutically effective amount of a serine hydrolase inhibitor (e.g., a lipase inhibitor) that results in the decrease in the activity of the serine hydrolase (e.g., lipase) outside the GI tract.

In some embodiments, the methods include prophylactically treating age-related diseases or conditions. As used herein, the term “prophylactically treating” can refer to a taking preventative measures to preserve health or prevent the progression of or occurrence of a disease or condition (e.g., reversing accumulation of a serine hydrolase or a lipase in an organ of a subject, reversing cellular damage in an organ of a subject, and/or preserving extracellular matrix in an organ of a subject). For example, a subject can be prophylactically treated when the subject is at risk of experiencing a disease or condition (e.g., having biomarkers that increase susceptibility of a particular condition, e.g., dementia).

### Subject

As used herein, the term “subject” refers an organism, typically a mammal (e.g., a human). In some embodiments, a subject is suffering from a relevant disease, disorder, or condition. In some embodiments, a subject is susceptible to a disease, disorder, or condition. In some embodiments, a subject displays one or more symptoms or characteristics of a disease, disorder, or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

In some embodiments, the subject can be an animal, human or non-human. Non-limiting examples of non-human subjects can include mice, rats, hamsters, rabbits, cats, dogs, horses, pigs, donkeys, monkeys, and/or other non-human primates such as apes and lemurs.

In some embodiments, the subject is a human. In some embodiments, a human patient can be an adult human or juvenile human (e.g., human below the age of 18 years old). In some embodiments, the subject is a patient suffering from an aging-related disease, disorder, or condition. In some embodiments, the subject is a patient susceptible to an aging-related disease, disorder, or condition. In some embodiments, the subject is a patient displaying one or more signs or symptoms or characteristics of an aging-related disease, disorder, or condition. In some embodiments, the subject is displaying symptoms of an age-related disease, disorder, or condition when the subject is considered biologically aged, e.g., over 50 years old, over 55 years old, over 60 years old, over 65 years old, over 70 years old, over 75 years old, over 80 years old, over 85 years old, over 90 years old, or over 95 years old. In some embodiments, the subject is displaying symptoms of an age-related disease, disorder, or condition at time when the subject is not considered biologically aged, e.g., under 45 years old, under 40 years old, under 35 years old, under 30 years old, or under 25 years old. In some embodiments, the subject is an adult human over the age of 18 years old. In some embodiments, the subject is older than 20 years old. In some embodiments, the subject is older than 30 years old. In some embodiments, the subject is older than 40 years old. In some embodiments, the subject is older than 50 years old. In some embodiments, the subject is older than 60 years old. In some embodiments, the subject is older than 70 years old. In some embodiments, the subject is older than 80 years old. In some embodiments, the subject is older than 90 years old. In some embodiments, the subject is older than 100 years old.

### Treating/Treatment

As used herein, the term “treating” means a reduction in the number, frequency, severity, or duration of one or more (e.g., two, three, four, five, or six) symptoms of a disease or disorder in a subject (e.g., any of the subjects described herein), and/or results in a decrease in the development and/or worsening of one or more symptoms of a disease or disorder in a subject.

As used herein, the term “therapeutically effective amount” means an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, stabilizes one or more characteristics of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. For example, in some embodiments, term “therapeutically effective amount”, refers to an amount which, when administered to an individual in need thereof in the context of inventive therapy, will block, stabilize, attenuate, or reverse aging-supportive process occurring in said individual, or will enhance or increase an aging-suppressive process in said individual.

A “therapeutically effective amount” of a composition described herein can reverse (in a therapeutic treatment) the development accumulation of a serine hydrolase (e.g., lipase) in an organ of a subject, reverse cellular damage in an organ of a subject, or preserve extracellular matrix structure in an organ of a subject. A therapeutically effective amount can include preserving the molecular structure of organ tissue as detected by hybridizing peptides that can bind to collagen structure at cleavage sites. A therapeutically effective amount administered to an individual to treat a disease or condition in that individual may be the same or different from a therapeutically effective amount administered for prophylactic purposes.

The therapeutic methods described herein are not to be interpreted as, restricted to, or otherwise limited to a “cure” for aging; rather the methods of treatment are directed to the use of the described compositions to “treat” age-related conditions, i.e., to effect a desirable or beneficial change in the health of an individual who has an age-related condition, such as but not limited to accumulation of a serine hydrolase (e.g., lipase) in an organ of a subject, reverse ongoing extracellular matrix protein (e.g., collagen) cleavage, cellular damage (e.g., membrane receptor cleavage) and cellular dysfunction (e.g., reduced integrin attachment to the extracellular matrix and intracellular integrin signaling) in an organ of a subject, or preserve extracellular matrix in an organ of a subject. As is understood in the art, an effective amount of a serine hydrolase inhibitor or a lipase inhibitor may vary, depending on, inter alia, patient history as well as other factors such as the type (and/or dosage) of serine hydrolase inhibitor or a lipase inhibitor used.

The phrases “reduced”, “decreased”, “a reduced level”, or “a decreased level” and similar phrases generally refer to a reduction or decrease of at least about 1% (e.g., about at least 2%, about at least 4%, about at least 6%, about at least 8%, about at least 10%, about at least 12%, about at least 14%, about at least 16%, about at least 18%, about at least 20%, about at least 22%, about at least 24%, about at least 26%, about at least 30%, about at least 35%, about at least 40%, about at least 45%, about at least 50%, about at least 55%, about at least 60%, about at least 65%, about at least 70%, about at least 75%, about at least 80%, about at least 85%, about at least 90%, about at least 95%, or about at least 99%) as compared to a reference level or value. The phrases “increased”, “greater”, “an increased level”, or “a greater level” and similar phrases generally refer to an increase of at least about 1% (e.g., about at least 2%, about at least 4%, about at least 6%, about at least 8%, about at least 10%, about at least 12%, about at least 14%, about at least 16%, about at least 18%, about at least 20%, about at least 22%, about at least 24%, about at least 26%, about at least 30%, about at least 35%, about at least 40%, about at least 45%, about at least 50%, about at least 55%, about at least 60%, about at least 65%, about at least 70%, about at least 75%, about at least 80%, about at least 85%, about at least 90%, about at least 95%, about at least 99%, about 100%, about 150%, about 200%, or more) as compared to a reference level or value.

In some embodiments, a therapeutically effective amount may be formulated and/or administered in a plurality of doses, for example, as part of a dosing regimen. In some embodiments, effective amounts and schedules for administering the hydrolase inhibitor or lipase inhibitor described herein may be determined empirically, and making such determinations is within the skill in the art. Those skilled in the art will understand that the dosage that must be administered will vary depending on, for example, the subject that will receive the hydrolase inhibitor or lipase inhibitor disclosed herein, the route of administration, the particular type of hydrolase inhibitor or lipase inhibitor, and other drugs being administered to the subject. In some embodiments, the administration of a therapeutically effective amount comprises chronic administration, whereas in other embodiments the administration of a therapeutically effective amount comprises a scheduled administration.

In some embodiments, the scheduled administration includes a predetermined schedule. Non-limiting examples of a scheduled basis include every other day, every two days, every three days, every four days, every five days, every six days, or once a week. Other non-limiting examples of a scheduled basis include one day on: six days off, two days on: five days off, three days on: four days off, four days on: three days off, five days on: two days off, six days on: one day off. Yet further non-limiting examples of a scheduled basis include two days on: one day off, two days on: two days off, two days on: three days off, two days on-four days off, two days on: five days off, three days on: one day off, three days on: two days off, three days on: three days off, three days on: four days off, four days on: one day off, four days on-two days off, four days on: three days off, five days on: one day off, five days on: two days off, six days on: one day off. Yet further non-limiting examples of a scheduled basis include one day out of every seven days, two days out of every seven days, three days out of every seven days, four days out of every seven days, five days out of every seven days, or six out of every seven days. The method may comprise administering the composition by a weekly protocol consisting of daily administration of a maintenance dose composition for 3-5 consecutive days followed by no administration for 1-3 consecutive days.

In some embodiments, the subject can be administered the hydrolase inhibitor or lipase inhibitor over an extended period of time (e.g., over a period of about at least 1 week, about 2 weeks, about 3 weeks, about 1 month, about 2 months, about 3 months, about 4 months, about 5 months, about 6 months, about 7 months, about 8 months, about 9 months, about 10 months, about 11 months, about 12 months, about 1 year, about 2 years, about 3 years, about 4 years, or about 5 years). A skilled medical professional may determine the length of the treatment period using any of the methods described herein for diagnosing or following the effectiveness of treatment (e.g., the observation of at least one symptom of aging). As described herein, a skilled medical professional can also change the identity and number (e.g., increase or decrease) of the hydrolase inhibitor or lipase inhibitor administered to the subject and can also adjust (e.g., increase or decrease) the dosage or frequency of administration of the hydrolase inhibitor or lipase inhibitor to the subject based on an assessment of the effectiveness of the treatment. In some embodiments, the hydrolase inhibitor or lipase inhibitor is administered for more than about 1 week. In some embodiments, the hydrolase inhibitor or lipase inhibitor is administered for more than about 2 weeks. In some embodiments, the hydrolase inhibitor or lipase inhibitor is administered for more than about 4 weeks. In some embodiments, the hydrolase inhibitor or lipase inhibitor is administered for about more than one month, about more than two months, about more than three months, about more than four months, about more than five months, about more than six months, about more than seven months, about more than eight months, about more than nine months, about more than 10 months, about more than 11 months, about more than 12 months, or longer.

In some embodiments, the hydrolase inhibitor or lipase inhibitor can be administered at a concentration that is lower than the serine protease or serine hydrolase (e.g., lipase) concentration within the GI tract. In some embodiments, the hydrolase inhibitor or lipase inhibitor can be administered at a concentration that is less than about 10% of the serine protease or serine hydrolase (e.g., lipase) concentration of the GI tract. In some embodiments, the hydrolase inhibitor or lipase inhibitor can be administered at a concentration that is less than about 9%, about 8%, about 7%, about 6%, about 5%, about 4%, about 3%, about 2%, or about 1% of the serine protease concentration or the serine hydrolase (e.g., lipase) concentration of the GI tract. For example, a small molecular weight serine hydrolase (e.g., lipase) competitive inhibitor (e.g., FUT-175), is administered at a concentration (e.g., 10 μM) below the serine protease or serine hydrolase (e.g., lipase) concentration inside the small intestine (e.g., 100 μM), but which matches and/or exceeds the serine protease or serine hydrolase (e.g., lipase) concentration in the plasma (e.g., 5 μM). In some embodiments, the hydrolase inhibitor or lipase inhibitor that is administered blocks the activity of serine proteases or serine hydrolases (e.g., lipases) in the plasma. In some embodiments, the majority of the digestive activity of the small intestine is preserved. In some embodiments, the concentration of the hydrolase inhibitor or lipase inhibitor does not interfere or reduce digestion or functional activity of the stomach and/or small intestine. The serine protease or serine hydrolase (e.g., lipase) concentration within the GI tract can be determined empirically or it may be determined by consultation to a standardized and accepted source of such information.

In some embodiments, the concentration of the hydrolase inhibitor or lipase inhibitor to be administered to a subject can be determined by measuring serine hydrolase (e.g., lipase) activity in the subject. In some embodiments, the concentration of the hydrolase inhibitor or lipase inhibitor to be administered to a subject can be determined by measuring the serine hydrolase (e.g., lipase) activity in the subject at a specific time point. In some embodiments, the concentration of the hydrolase inhibitor or lipase inhibitor to be administered to a subject can be determined by measuring the or serine hydrolase (e.g., lipase) activity outside the GI tract (e.g., in the plasma, in the peripheral tissue) of the subject. In some embodiments, the serine hydrolase (e.g., lipase) within the GI tract is determined by mass spectrometry determination of peptide incidence in plasma. For example, a sample of a patient's plasma can be run through a mass spectrometer and proteolysis of the plasma proteins can be determined. In some embodiments, the serine hydrolase (e.g., lipase) concentration can be determined by receptor cleavage with antibody against extracellular domains using cells harvested from the subject or the subject's plasma or other body fluid (e.g., lymph fluid).

In some embodiments, the hydrolase inhibitor or lipase inhibitor can be administered at a concentration that is lower than the serine protease or serine hydrolase (e.g., lipase) concentration within the GI tract but higher than the serine protease or serine hydrolase (e.g., lipase) concentration outside the GI tract (e.g., in plasma, or in peripheral tissues). In some embodiments, the hydrolase inhibitor or lipase inhibitor can be administered at a concentration that is about the same as the serine protease concentration or serine hydrolase (e.g., lipase) outside the GI tract. In some embodiments, the hydrolase inhibitor or lipase inhibitor can be administered at a concentration that is higher than the hydrolase inhibitor or lipase inhibitor concentration outside the GI tract. In some embodiments, the hydrolase inhibitor or lipase inhibitor can be administered at a concentration that is at least about 60%, at least about 65%, at least about 70%, at least about 75%, at least about 80%, at least about 85%, at least about 90%, at least about 95%, or about 100% the concentration of the serine hydrolase (e.g., lipase) in the subject's plasma and/or tissue.

In some embodiments, the hydrolase inhibitor or lipase inhibitor can be administered at a concentration of less than about 10 mM, less than about 9 mM, less than about 8 mM, less than about 7 mM, less than about 6 mM, less than about 5 mM, less than about 4 mM, less than about 3 mM, less than about 2 mM, less than about 1 mM, less than about 0.5 mM, or less than about 0.1 mM.

In some embodiments, the hydrolase inhibitor or lipase inhibitor can be administered at a concentration of less than about 50 μM (e.g., less than about 48 μM, less than about 46 μM, less than about 44 μM, less than about 42 μM, less than about 40 μM, less than about 38 μM, less than about 36 μM, less than about 34 μM, less than about 32 μM, less than about 30 μM, less than about 28 μM, less than about 26 μM, less than about 24 μM, less than about 22 μM, less than about 20 μM, less than about 18 μM, less than about 16 μM, less than about 14 μM, less than about 12 μM, less than about 10 μM, less than about 8 μM, less than about 6 μM, less than about 5 μM, less than about 4 μM, less than about 3 μM, less than about 2 μM, less than about 1 μM, less than about 0.5 μM, less than about 0.25 μM, or less than about 0.1 μM). In some embodiments, the hydrolase inhibitor or lipase inhibitor is administered at a concentration of less than about 5 μM. In some embodiments, the hydrolase inhibitor or lipase inhibitor is administered at a concentration of less than about 1 μM (e.g., less than about 0.8 μM, less than about 0.6 μM, less than about 0.4 μM, less than about 0.2 μM, less than about 0.1 μM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 3 nM, less than about 1 nM, less than about 0.8 nM, less than about 0.6 nM, less than about 0.4 nM, less than about 0.2 nM, less than about 0.1 nM, less than about 90 pM, less than about 80 pM, less than about 70 pM, less than about 60 pM, less than about 50 pM, less than about 40 pM, less than about 30 pM, less than about 20 pM, less than about 10 pM, less than about 5 pM, less than about 3 pM, less than about 1 pM, less than about 0.8 pM, less than about 0.6 pM, less than about 0.4 pM, less than about 0.2 pM, or less than about 0.1 pM).

In some embodiments, the hydrolase inhibitor or lipase inhibitor can be administered according to the patient's weight. In some embodiments the hydrolase inhibitor or lipase inhibitor can be administered anywhere between about 0.01 and about 1.0 gm/kg/day. In some embodiments, a therapeutically effective amount of hydrolase inhibitor or lipase inhibitor can include about 0.01 gm/kg/day, about 0.02 gm/kg/day, about 0.03 gm/kg/day, about 0.04 gm/kg/day, about 0.05 gm/kg/day, about 0.06 gm/kg/day, about 0.07 gm/kg/day, about 0.08 gm/kg/day, about 0.09 gm/kg/day, about 0.1 gm/kg/day, about 0.11 gm/kg/day, about 0.12 gm/kg/day, about 0.13 gm/kg/day, about 0.14 gm/kg/day, about 0.15 gm/kg/day, about 0.16 gm/kg/day, about 0.17 gm/kg/day, about 0.18 gm/kg/day, about 0.19 gm/kg/day, about 0.20 gm/kg/day, about 0.21 gm/kg/day, about 0.22 gm/kg/day, about 0.23 gm/kg/day, about 0.24 gm/kg/day, about 0.25 gm/kg/day, about 0.26 gm/kg/day, about 0.27 gm/kg/day, about 0.28 gm/kg/day, about 0.29 gm/kg/day, about 0.30 gm/kg/day, about 0.31 gm/kg/day, about 0.32 gm/kg/day, about 0.33 gm/kg/day, about 0.34 gm/kg/day, about 0.35 gm/kg/day, about 0.36 gm/kg/day, about 0.37 gm/kg/day, about 0.38 gm/kg/day, about 0.39 gm/kg/day, about 0.40 gm/kg/day, about 0.41 gm/kg/day, about 0.42 gm/kg/day, about 0.43 gm/kg/day, about 0.44 gm/kg/day, about 0.45 gm/kg/day, about 0.46 gm/kg/day, about 0.47 gm/kg/day, about 0.48 gm/kg/day, about 0.49 gm/kg/day, about 0.50 gm/kg/day, about 0.51 gm/kg/day, about 0.52 gm/kg/day, about 0.53 gm/kg/day, about 0.54 gm/kg/day, about 0.55 gm/kg/day, about 0.56 gm/kg/day, about 0.57 gm/kg/day, about 0.58 gm/kg/day, about 0.59 gm/kg/day, about 0.60 gm/kg/day, about 0.61 gm/kg/day, about 0.62 gm/kg/day, about 0.63 gm/kg/day, about 0.64 gm/kg/day, about 0.65 gm/kg/day, about 0.66 gm/kg/day, about 0.67 gm/kg/day, about 0.68 gm/kg/day, about 0.69 gm/kg/day, about 0.70 gm/kg/day, about 0.71 gm/kg/day, about 0.72 gm/kg/day, about 0.73 gm/kg/day, about 0.74 gm/kg/day, about 0.75 gm/kg/day, about 0.76 gm/kg/day, about 0.77 gm/kg/day, about 0.78 gm/kg/day, about 0.79 gm/kg/day, about 0.80 gm/kg/day, about 0.81 gm/kg/day, about 0.82 gm/kg/day, about 0.83 gm/kg/day, about 0.84 gm/kg/day, about 0.85 gm/kg/day, about 0.86 gm/kg/day, about 0.87 gm/kg/day, about 0.88 gm/kg/day, about 0.89 gm/kg/day, about 0.90 gm/kg/day, about 0.91 gm/kg/day, about 0.92 gm/kg/day, about 0.93 gm/kg/day, about 0.94 gm/kg/day, about 0.95 gm/kg/day, about 0.96 gm/kg/day, about 0.97 gm/kg/day, about 0.98 gm/kg/day, about 0.99 gm/kg/day, about 1.0 gm/kg/day.

In some embodiments, depending on the properties of the hydrolase inhibitor or lipase inhibitor, a therapeutically effective amount of hydrolase inhibitor or lipase inhibitor can include amounts higher than about 1.0 gm/kg/day. For example, a therapeutically effective amount of hydrolase inhibitor or lipase inhibitor can include, about **about 1 gm/kg/day, about 2 gm/kg/day, about 3 gm/kg/day, about 4 gm/kg/day, about 5 gm/kg/day, about 6 gm/kg/day, about 7 gm/kg/day, about 8 gm/kg/day, about 9 gm/kg/day, about 10 gm/kg/day, about 11 gm/kg/day, about 12 gm/kg/day, about 13 gm/kg/day, about 14 gm/kg/day, about 15 gm/kg/day, about 16 gm/kg/day, about 17 gm/kg/day, about 18 gm/kg/day, about 19 gm/kg/day, about 20 gm/kg/day, about 21 gm/kg/day, about 22 gm/kg/day, about 23 gm/kg/day, about 24 gm/kg/day, about 25 gm/kg/day, about 26 gm/kg/day, about 27 gm/kg/day, about 28 gm/kg/day, about 29 gm/kg/day, about 30 gm/kg/day, about 31 gm/kg/day, about 32 gm/kg/day, about 33 gm/kg/day, about 34 gm/kg/day, about 35 gm/kg/day, about 36 gm/kg/day, about 37 gm/kg/day, about 38 gm/kg/day, about 39 gm/kg/day, about 40 gm/kg/day, about 41 gm/kg/day, about 42 gm/kg/day, about 43 gm/kg/day, about 44 gm/kg/day, about 45 gm/kg/day, about 46 gm/kg/day, about 47 gm/kg/day, about 48 gm/kg/day, about 49 gm/kg/day, about 50 gm/kg/day, about 51 gm/kg/day, about 52 gm/kg/day, about 53 gm/kg/day, about 54 gm/kg/day, about 55 gm/kg/day, about 56 gm/kg/day, about 57 gm/kg/day, about 58 gm/kg/day, about 59 gm/kg/day, about 60 gm/kg/day, about 61 gm/kg/day, about 62 gm/kg/day, about 63 gm/kg/day, about 64 gm/kg/day, about 65 gm/kg/day, about 66 gm/kg/day, about 67 gm/kg/day, about 68 gm/kg/day, about 69 gm/kg/day, about 70 gm/kg/day, about 71 gm/kg/day, about 72 gm/kg/day, about 73 gm/kg/day, about 74 gm/kg/day, about 75 gm/kg/day, about 76 gm/kg/day, about 77 gm/kg/day, about 78 gm/kg/day, about 79 gm/kg/day, about 80 gm/kg/day, about 81 gm/kg/day, about 82 gm/kg/day, about 83 gm/kg/day, about 84 gm/kg/day, about 85 gm/kg/day, about 86 gm/kg/day, about 87 gm/kg/day, about 88 gm/kg/day, about 89 gm/kg/day, about 90 gm/kg/day, about 91 gm/kg/day, about 92 gm/kg/day, about 93 gm/kg/day, about 94 gm/kg/day, about 95 gm/kg/day, about 96 gm/kg/day, about 97 gm/kg/day, about 98 gm/kg/day, about 99 gm/kg/day, or about 100 gm/kg/day.

### Administration

As used herein, the term “administration” typically refers to the administration of a composition to a subject or system to achieve delivery of an agent that is, or is included in, the composition. Those of ordinary skill in the art will be aware of a variety of routes that may, in appropriate circumstances, be utilized for administration to a subject, for example a human. For example, in some embodiments, administration may be ocular, oral, enteral, parenteral, etc. In some particular embodiments, administration may be bronchial (e.g., by bronchial instillation), buccal, enteral, intra-arterial, intragastric, intramedullary, intramuscular, intranasal, intraperitoneal, intrathecal, intravenous, intraventricular, intracisternal, within a specific organ (e.g., intrahepatic), mucosal, nasal, oral, rectal, subcutaneous, sublingual, topical, tracheal (e.g., by intratracheal instillation), vaginal, vitreal, by patch, etc. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time. In some embodiments, administration may involve methods of delivery that include, but are not limited to, use of external and/or implanted infusion pumps, liquid formulation, capsulated formulation, or slow release encapsulation.

In some embodiments, the hydrolase inhibitor or lipase inhibitor administration can be ocular, oral, parenteral, bronchial (e.g., by bronchial instillation), buccal, enteral, intra-arterial, intragastric, intramedullary, intramuscular, intranasal, intraperitoneal, intrathecal, intravenous, intraventricular, intracisternal, within a specific organ (e.g., intrahepatic), mucosal, nasal, oral, rectal, subcutaneous, sublingual, tracheal (e.g., by intratracheal instillation), vaginal, or vitreal. In some embodiments, the hydrolase inhibitor or lipase inhibitor is administered by enteral administration, intraperitoneal administration, intravenous administration, intramuscular administration, subcutaneous administration, intracutaneous administration, oral administration, intranasal administration, intrapulmonary administration, intrarectal administration, or a telemetry controlled external or implanted infusion pump. In some embodiments, the hydrolase inhibitor or lipase inhibitor is administered by oral administration. In some embodiments, the hydrolase inhibitor or lipase inhibitor is administered by a telemetry controlled infusion pump. In some embodiments, the telemetry controlled infusion pump is directed toward a target tissue. In some embodiments, the target tissue can include adipose tissue, pancreatic tissue, liver tissue, kidney tissue, lung tissue, vasculature, bone tissue, central nervous system (CNS) tissue, eye tissue, muscle tissue, and secondary lympho-organ tissue. In some embodiments, the target tissue can include veins, arteries, lymphatics, cerebral spinal fluid, subcutaneous tissue, or joints. In some embodiments, the telemetry controlled infusion pump is directed toward a target organ. In some embodiments, the target organ can include the brain, spinal cord, heart, kidney, muscle, liver, lung, pancreas, or any other organ. In some embodiments, the hydrolase inhibitor or lipase inhibitor is administered in a form of eye drops or liposome composition.

In some embodiments, the subject can be administered more than one hydrolase inhibitor or lipase inhibitor. In some embodiments, the administration of the one or more hydrolase inhibitors or lipase inhibitors is sequential administration (e.g., one lipase inhibitor is administered, stopped, and a second, different lipase inhibitor or other hydrolase inhibitor is administered). In some embodiments, the subject can be administered a combination of serine hydrolase inhibitors at the same time.

In some embodiments, the methods of the disclosure can be administered before, in conjunction with, or after other methods or therapeutic treatments, either for the condition being treated by administration of the hydrolase inhibitor or lipase inhibitor, or another condition. In a non-limiting example, a subject can be treated with surgery for a cancerous mass in the intestine, and after the surgery the subject can be administered a hydrolase inhibitor or lipase inhibitor to limit leakage of proteases from the intestine. Preventative/prophylactic administration of hydrolase inhibitors or lipase inhibitors can be used to slow and/or prevent autodigestion of the subject's organs due to the intestinal permeability. In another non-limiting example, the subject may be administered a hydrolase inhibitor or lipase inhibitor for treatment of suspected dementia, while the subject is concurrently taking another medication either for the subject's dementia or for another condition (e.g., diabetes).

In some embodiments, serine protease activity is increased in a postprandial period. To block this activity, the hydrolase inhibitor or lipase inhibitor can be administered before food intake (e.g., eating). In diabetics or pre-diabetics, this postprandial period of elevated serine protease activity is longer than in non-diabetics, and may last for several hours. In some embodiments, the amount or concentration of hydrolase inhibitor or lipase inhibitor administration will depend on measurements of protease activity in plasma or in peripheral tissues, like abdominal fluid, heart, brain, intestine, kidney, liver, lung, eye, or other tissues disclosed herein. In some embodiments, the hydrolase inhibitor or lipase inhibitor is administered during a diurnal cycle, wherein the hydrolase inhibitor or lipase inhibitor administration depends on the measured serine protease activity in the subject.

### Organs/Conditions/Diseases

In some embodiments, the method provided herein can reduce symptoms of biological aging in a subject. In some embodiments, the subject can display one or more signs or symptoms or characteristics of an aging-related disease, disorder, or condition.

In some embodiments, the methods involved selecting a subject at risk of damage to an organ. The subject can be at risk of damage to the organ because the subject is exhibiting symptoms consistent with a known disease or condition that affects that organ. The subject can be at risk of damage to the organ because the subject has a biomarker known to predispose the subject to a known disease or condition that affects that organ. The subject can be at risk of damage to the organ because the subject has a family history that would predispose them to a known disease or condition that affects that organ. In some embodiments, the subject demonstrates symptoms and/or biomarkers of elevated serine protease activity outside the GI tract (e.g., in plasma, or in peripheral tissue).

For the methods disclosed herein, the organ may be one or more of the brain, spinal cord, heart, kidney, muscle, intestine, liver, eye, skeletal muscle, abdominal and subcutaneous adipose tissue, small and large intestinal wall, connective tissue (including mesentery), breast tissue, tissues from the male and female reproductive organs, bone, cartilage, car, nasal tract, and lung. In some embodiments, the organ may be one or more of brain, heart, intestine, and muscle. In some embodiments, the organ is the heart. In some embodiments the organ is the brain. In some embodiments, the organ is the kidney. In some embodiments, the organ is the liver. In some embodiments, the organ is the intestine (e.g., small intestine and/or large intestine). In some embodiments, the organ is the eye. In some embodiments, the organ is the lung. For clarity, the methods of the disclosure are not intended to treat shock (e.g., septic shock) or HIV. Further, the methods of the disclosure may further include a step of screening a subject for shock (e.g., septic shock) and/or HIV, and not administering a hydrolase inhibitor or lipase inhibitor to the subject if the subject is currently experiencing shock or biomarkers of HIV.

In some embodiments, the aging-related disease can include mild cognitive impairment, Alzheimer's disease, age-related loss of neuronal function including but not limited to memory loss, loss of balance, and sensory function, pain, including but not limited to lower back pain, aneurysm, chronic venous disease, cystic fibrosis, fibrosis in pancreatitis, glaucoma, hypertension, idiopathic pulmonary fibrosis, inflammatory bowel disease, intervertebral disc degeneration, osteoarthritis, type 2 diabetes mellitus, adipose atrophy, lipodystrophy, atherosclerosis, cataracts, COPD, kidney transplant failure, liver fibrosis, loss of bone mass, myocardial infarction, sarcopenia, wound healing, alopecia, cardiomyocyte hypertrophy, osteoarthritis, Parkinson's disease, age-associated loss of lung tissue elasticity, age-related macular degeneration, cachexia, glomerulosclerosis, liver cirrhosis, nonalcoholic fatty liver disease (NAFLD), nonalcoholic steatohepatitis (NASH), hepatitis A, hepatitis B, hepatitis C, autoimmune hepatitis, primary biliary cholangitis, primary sclerosing cholangitis, hemochromatosis, Wilson's disease, alpha-1 antitrypsin deficiency, liver cancer, bile duct cancer, liver adenoma, osteoporosis, Huntington's disease, spinocerebellar ataxia, mental disorders, neurodegeneration, epilepsy or other seizure disorders, stroke, cancer, dementia, including frontomemporal dementia, meningitis, encephalitis, vascular disease, coronary heart disease, angina, unstable angina, heart failure, valve disease, high blood pressure, heart arrhythmias, endocardidtis, pericardial disease, cardiomyopathy, infection susceptibility, chronic inflammation, renal dysfunction, rheumatoid arthritis, inflammatory bowel disease, lupus erythematosus, lupus nephritis, diabetic nephropathy, CNS injury, amyotrophic lateral sclerosis, fibromyalgia, myositis, including polymyositis and dermatomyositis, muscular dystrophy, myasthenia gravis, rhabdomyolysis, sarcopenia, Charcot-Marie-Tooth disease, myopathy, peripheral neuropathy, and spinal muscular atrophy, acute kidney injury, kidney stones, kidney infections, including pyelonephritis, kidney cysts, kidney cancer, conditions where the subject is in need of renal dialysis, pancreatic cancer, Crohn's disease, multiple sclerosis, Guillain-Barre syndrome, psoriasis, Grave's disease, ulcerative colitis, mood disorders and/or cognitive impairment.

### Kits

Also provided herein are kits for the use in the methods described herein. For example, the kids can include a composition comprising a hydrolase inhibitor or lipase inhibitor for oral administration. Instructions for use can also be included in the kits.

In some embodiments, the pharmaceutical compositions described herein may be provided in a kit. In such aspects, the kit may comprise a syringe prefilled with the pharmaceutical composition, a hypodermic syringe, and instructions for use. In some embodiments, the pharmaceutical compositions may be provided as a single or multiple dose pharmaceutical composition in a pre-filled syringe. In some embodiments, the pharmaceutical compositions may be provided as a single or multiple dose pharmaceutical composition in a vial. In some embodiments, the pharmaceutical composition is suitable for diagnostic or therapeutic use in vitro, in vivo, and/or ex vivo.

### EXAMPLES

### Example 1: Sample Collection and Serine Protease Inhibition in Various Organs in Rats

### Animals and Tissue Collection

Male Wistar rats (Harlan Sprague Dawley Inc., Indianapolis, IN) at maturity (4 months, 300 to 350 gm) and at old age (24 months, 375 to 450 gm) were included in the study. The animals were maintained on standard laboratory chow (8604 Teklad rodent diet; Harlan Laboratories, Indianapolis, Ind) without restriction and water ad libitum and maintained in a separate room without pathogen-free conditions. They were confirmed to exhibit normal mobility, water and food consumption, and fecal material discharge. Animals that exhibited signs of morbidities were excluded. A subgroup of old animals was given a serine protease inhibitor (tranexamic acid, 14 days) in drinking water (137 mM, exchanged daily) which at a minimum fluid consumption of 40 ml/day amounts to a minimum dose of 0.39 gm/kg/day.

A femoral venous catheter was placed after general anesthesia (pentobarbital sodium, 50 mg/kg [Abbott Laboratories, North Chicago, Il1], intramuscularly after local anesthesia [2% lidocaine HCl; Hospira, Inc, Lake Forrest, Il1]). Tissues (intestine, liver, lung, heart, kidney, brain, mesentery, skin) were immediately collected after euthanasia (Beuthanasia i.v., 120 mg/kg, Schering-Plough Animal Health Corp, Union, NJ), fixed (formalin, 10%, neutral buffered, 1 hr), postfixed (fresh formalin, 24 hrs), and stored (formalin, 10%). The period between initial anesthesia and fixation of the tissues was below 60 minutes. All tissues were excised with sharp blades to minimize the stretching of collagen before fixation.

### Tissue Sections

Formalin-fixed tissues were cut into sections with a vibratome (thickness 40 μm; Pelco® Lancer™ Vibratome® Series 1000). The areas of the section were kept above ˜3 mm×˜5 mm to permit analysis of digestive enzyme infiltration over diverse regions within an organ.

To generate thin sections for the intestine, a segment of the upper jejunum was embedded in resin (Araldite; Polysciences, Washington, PA) and cut into 1 μm sections (Ultramicrotome, LKB Ultratome Nova). The resin was removed with Maxwell solution (2 g KOH in 10 ml absolute methyl alcohol+5 ml propylene oxide), rinsed in tap water, incubated in hydrogen peroxide (4%, 1 minute), rinsed (phosphate buffer), and immunolabeled for trypsin (see below).

### Digestive Enzyme Immunohistochemistry

To determine on the tissue sections the immunolabel density and distribution of digestive enzymes, the following primary antibodies were used: pancreatic trypsin MoAb (D-1): sc-137077 (Santa Cruz); pancreatic elastase (ELA1) polyclonal antibody (Biomatik®); pancreatic lipase MoAb (A-3): sc-374612 (Santa Cruz Biotechnology®); amylase MoAb (G-10): sc-46657 (Santa Cruz Biotechnology®). Primary antibodies were diluted to 1-1.5 μl/1000 μl of phosphate-buffered saline. They were followed by secondary antibodies (MP-7601 for anti-rabbit IgG; MP-7602 for anti-mouse IgG; ImmPRESS Excel staining kit peroxidase). Two substrate colors were used, red (ImmPACT™ AEC Substrate kit peroxidase, sk-4205; Vector® Laboratories) and brown (ImmPACT™ DAB [3,3′-diaminobenzidine] Substrate kit peroxidase, sk4105; and Vectorstain Elite ABC-HRP Kit, Vector® Laboratories). Sections without primary antibodies served as controls. No counterstain was applied to facilitate quantitative label intensity measurements. The concentrations and exposure of primary and secondary antibodies applied to the sections were adjusted (24 hrs and according to protocol by Vector® Laboratories, respectively) to achieve full penetration of the antibodies into the tissue sections. For each tissue, the labeling procedures were standardized among the animal groups to permit a quantitative comparison of label densities between ages and treatment with digital image analysis.

### Whole Mount Tissue Labeling

Small intestine: Full-thickness tissue blocks of the wall of the proximal jejunum (3×5 mm) were fixed from all sides in 10% formalin. Digestive enzymes were detected with primary and secondary antibodies labeled with 3, 3′-diaminobenzidine (DAB, Peroxidase Substrate Kit, ab64238, Abcam®).

The mucin-containing layer on the epithelial cells of the small intestine was stained using alcian blue (pH 2.5, kt 003; Diagnostic BioSystems, Pleasanton, CA) followed by a rinse in distilled water and mounted on a microscope slide (Vector Mount AQ Aqueous Mounting Medium, Vector® Laboratories, Burlington, CA).

To co-label the small intestine for mucin and trypsin, the fixed intestine was immersed in the primary antibody against trypsin and stained with DAB substrate. Thereafter the tissue section was embedded in resin and sectioned into thin (1 μm) sections. The mucin label (alcian blue), was applied to the thin section, coverslipped, and imaged.

Mesentery: The trypsin distribution in intact mesentery sectors was delineated by biotin/avidin immunolabeling with MoAB (D-1), secondary antibody (anti-mouse IgG, MP-7602, ImmPRESS™ Excel staining kit peroxidase, Vector® Laboratories) with a brown substrate (ImmPACT™ DAB sk-4105).

### Collagen Fragment Labeling

To localize molecular level subfailure of collagen with specificity, sections were labeled with biotin-conjugated collagen hybridizing peptides (B-CHP) that bind unfolded collagen by triple helix formation. The trimeric CHP are thermally dissociated to monomers before use (80° C. for 10 min), the hot CHP solution is quickly cooled to room temperature (by immersion into 4° C. water for 15 sec), diluted (1 μl in 1000 μl phosphate buffer saline, applied solution 7.5 mM) and immediately applied to the section (dead time <1 min). In this way, most CHP peptides are expected to remain as active monomers during the staining process, based on kinetic studies on CHP triple helix folding. Sections are incubated overnight at room temperature, and unbound B-CHP is washed (3 times in 1 ml of 1×PBS for 30 min at room temperature). To visualize the B-CHP, the tissue sections are incubated with streptavidin peroxidase (sk-5704, Vector® Laboratories, according to manufacturer instructions) and then to a substrate (ImmPact™ AEC Substrate Kit Peroxidase; sk-4205, Vector® Laboratories) at room temperature (for periods between 1 and 10 min depending on the tissue). The B-CHP label intensity on the sections is recorded by digital brightfield microscopy (40×, numerical aperture 0.5).

### Glucose Analysis

At the time of tissue collection, fresh femoral arterial blood was used to measure the blood glucose level (Contour®, Bayer® Diabetes Care, Tarrytown, NY) and the percent of glycated hemoglobin levels (AIC Home Test; Bristol-Myers Squibb®; NY, NY).

### Brain Insulin Receptor Density

Measurements of insulin receptor density were carried out by immunolabeling its extracellular domain on fixed tissue sections (10% formalin, neutral buffered) with a primary antibody (M-20, sc-57344 HRP, monoclonal antibody mapping to the N-terminus, Santa Cruz Biotechnology®) and visualize with a substrate (ImmPACT™ AEC Substrate kit peroxidase, SK-4205, Vector® Laboratories). Sections without primary antibodies were used as negative controls.

### Digital Image Analysis

Images of the immunolabel density were recorded at multiple magnifications (between 10× objective, numerical aperture 0.25, and 60×, numerical aperture 1.4). They were recorded under standard light conditions with fixed optical and digital camera settings (SPOT Insight® GIGABIT camera, Sterling Height) so that the camera serves as a quantitative light intensity meter without pixel intensity saturation. Images were analyzed digitally (Photoshop®, Adobe® 24.4.1.; spatial resolution of 640×480 pixels).

The red color of the biotin-conjugated collagen hybridizing peptides and the red immune substrates was digitally extracted and their intensity was measured on a black and white (B/W) scale (1 to 256 digital units between white and black, respectively). The density of the immune substrate label was measured in the form of digital light intensity (I) at a constant incident light intensity (Iₒ) without a tissue section.

Insulin receptors' densities on random tissue sections, labeled with an antibody against the extracellular domain, are digitally recorded by placing an optical window on the cell and determining light intensity at a constant incident light Iₒ.

Unless specified otherwise, the mean label density per group (3 animals/group) is determined from the average label density per animal (5 tissue sections/animal, ˜10 images/section).

### Statistics

Measurements are summarized as mean±standard deviation. For comparisons between young and old, an unpaired two-tailed Student's t-test was used. Analysis of variance (ANOVA) was used to test for differences in outcomes of interest among groups. Results were determined to be significant at p<0.05. Bonferroni's post hoc multiple comparison test was used to determine the significance between individual groups. To obtain statistically conclusive results, the minimum number of animals was estimated assuming equal variances among groups, α=0.05 and β=1-0.9. No animals were excluded from the analysis.

### Example 2: Digestive Enzyme Accumulation in Old Organs Outside the Gastrointestinal Tract

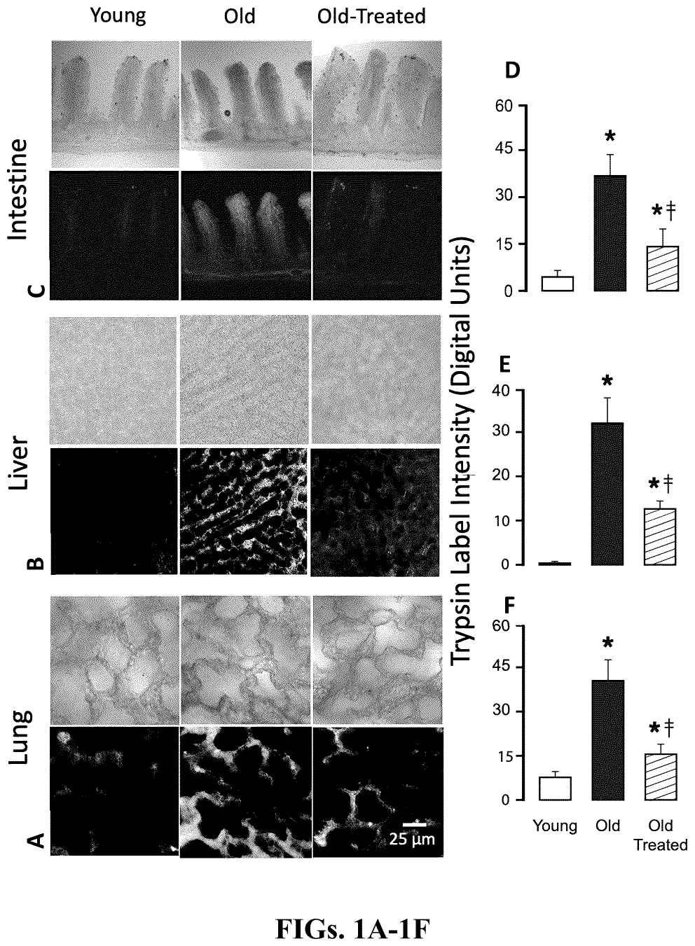

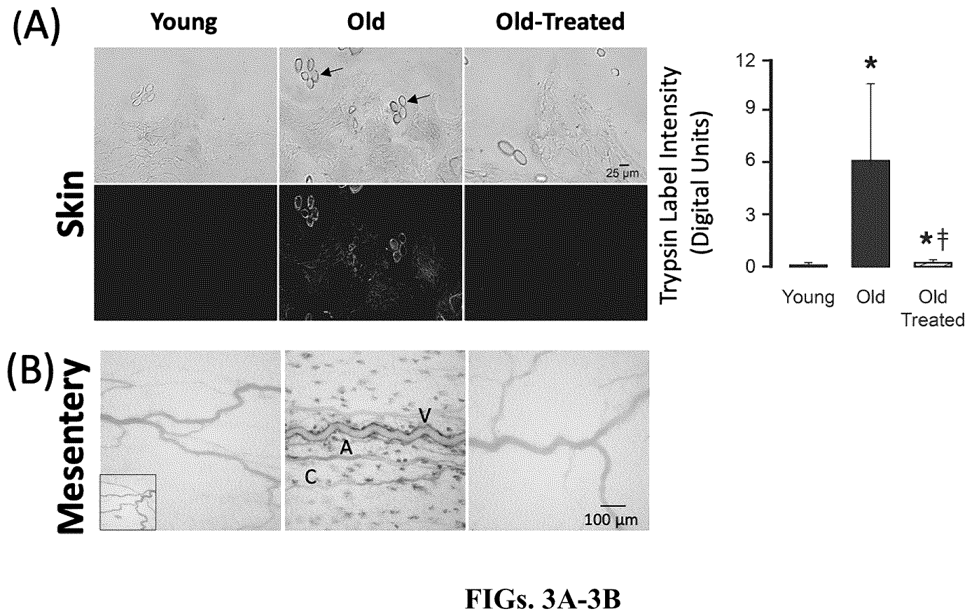

In young rats, all tissues in this study (intestinal wall, mesentery, liver, lung, heart, kidney, brain, skin) (FIGS. 1A-1F, 2A-2F, and 3A-3B) exhibit low immunolabel density for pancreatic trypsin. The villi of the small intestine and the lung tissue, compared to other tissues of the young, have a slightly enhanced trypsin label density.

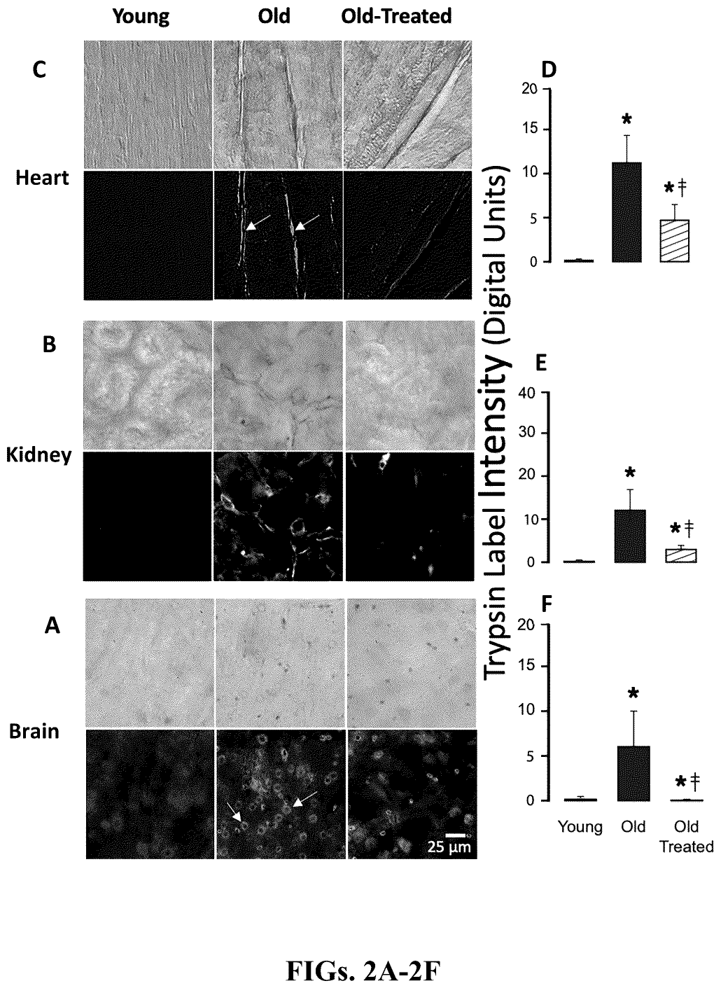

In contrast, the tissues of old rats have a significantly increased trypsin label density (FIGS. 1A-1F, 2A-2F, and 3A-3B). High densities are on sections of the intestine, liver, and lung, organs that are in the pathway of digestive enzymes leaking from the small intestine, including the venules of the mesentery (FIG. 1C. Trypsin labels are enriched in extracellular spaces (e.g. between heart muscle cells), in the wall of capillaries (e.g. brain), and in the follicles of the skin (FIG. 2C, arrows).

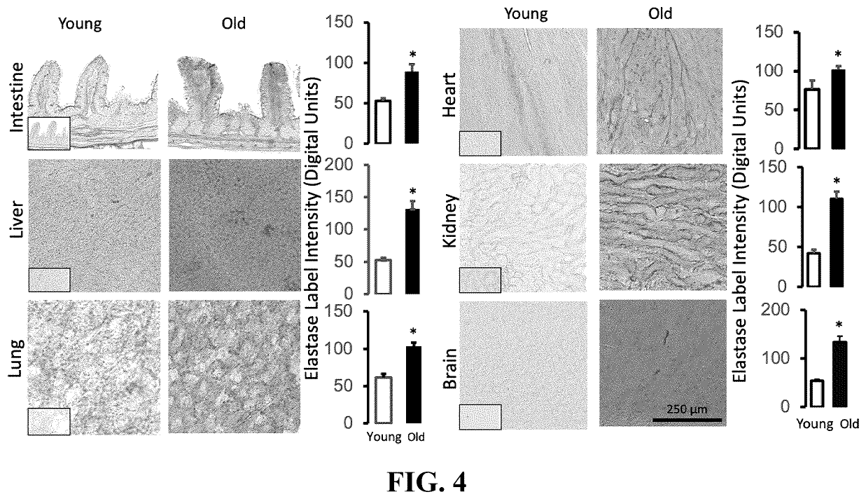

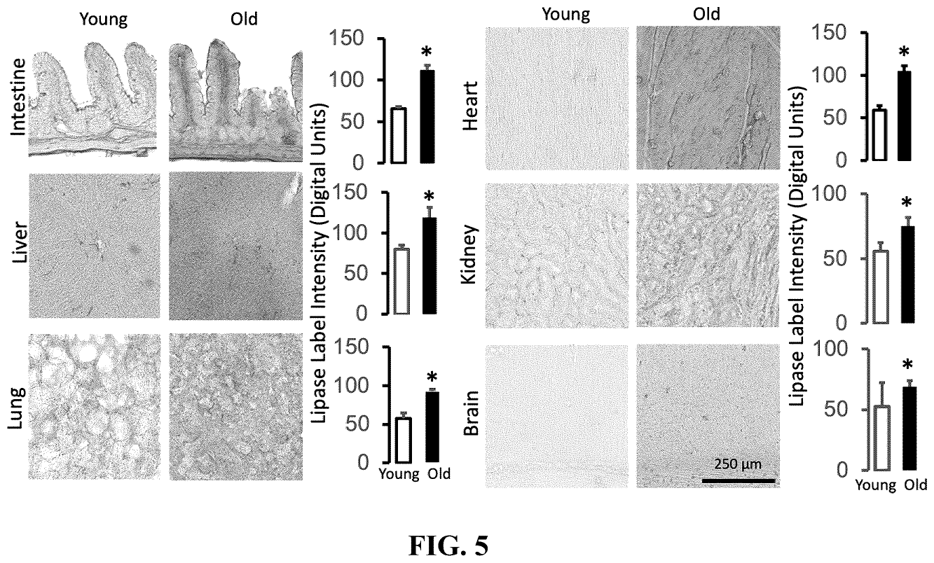

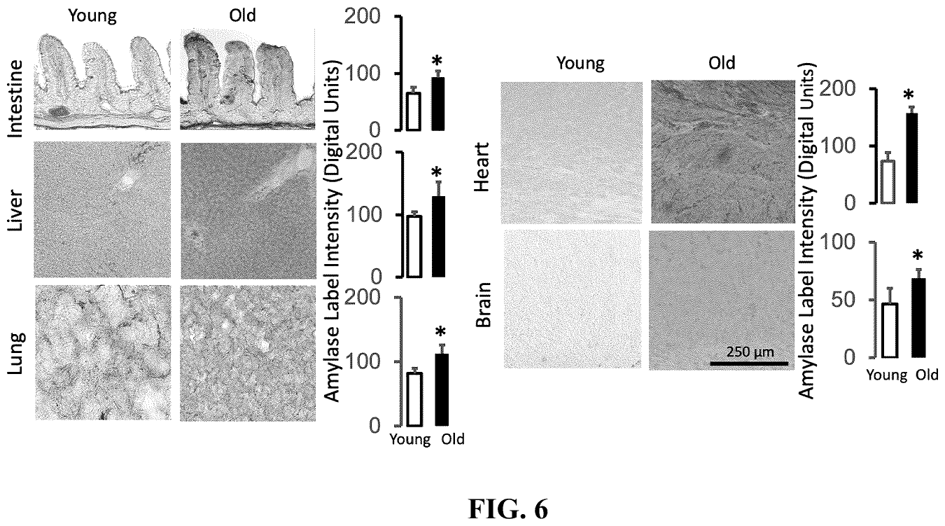

Pancreatic elastase, lipase, and amylase also exhibit low immunolabel densities in young tissues, that is increased in the old (FIGS. 4-6). The labeling pattern of these pancreatic enzymes is also tissue type specific (e.g. with interstitial accumulation between myocytes or in the microvasculature of the brain). However, the average label density was relatively uniform within each old organ as seen by the label density variances (<10%) across individual tissue sections (FIGS. 4-6, histograms). The measurements suggest that key pancreatic digestive enzymes have uniformly infiltrated the vital organs outside the pancreas and the lumen of the small intestine of old rats.

Two-week treatment of the old rats with oral small molecular weight trypsin inhibitor served to significantly reduce the accumulation of trypsin in these vital organs and the skin (FIGS. 1A-1F, 2A-2F, and 3A-3B; old-treated groups) although not to the low level of the young. An exception is the brain, which exhibited trypsin label density in old-treated rats that almost reached the level in the young (no significant difference). After the treatment of old rats, low trypsin label densities were observed in the mesentery that were not different from the young (FIGS. 1C-1D).

### Example 3: Intestinal Mucin-Epithelial Barrier and Digestive Enzyme Accumulation in the Small Intestine

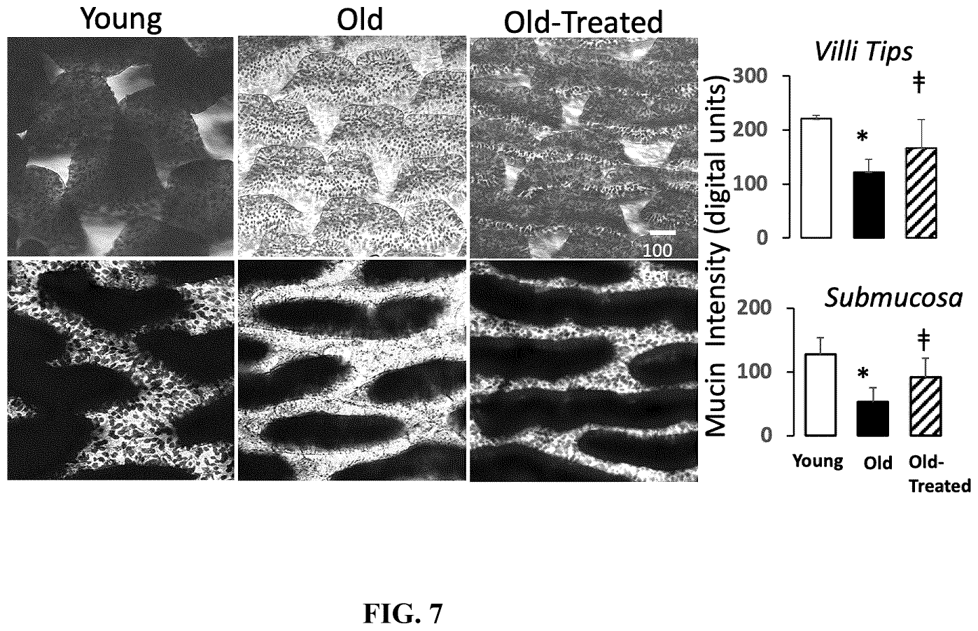

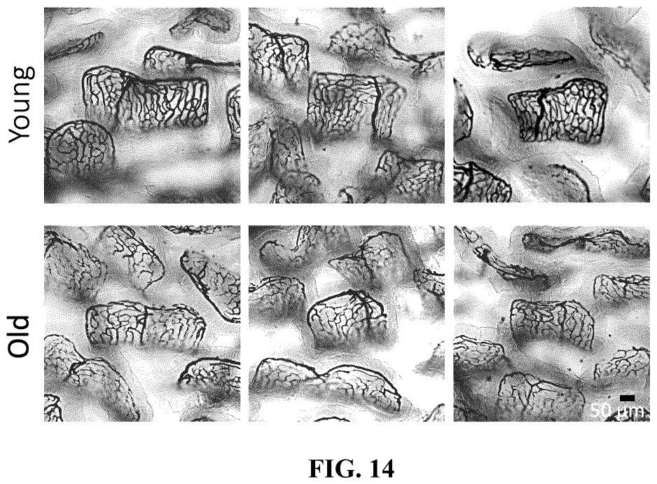

The villi in the upper jejunal segment of the rat small intestine had an elongated crest shape, with an alignment parallel to the long axis of the intestine (FIG. 7). The capillary network inside the villi crests was preserved in the old (FIG. 14).

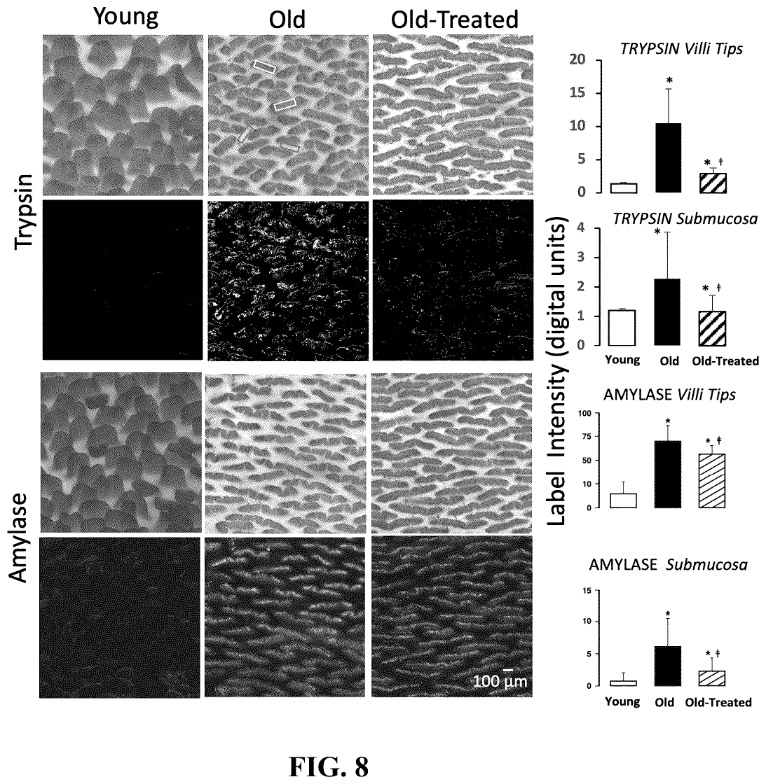

The mucin layer on the epithelium of the small intestine, a barrier for digestive enzymes, had significantly reduced density in the aged, both on and between villi crests (FIG. 7). Co-labeling of mucins and pancreatic trypsin or amylase in the intestine demonstrated that the reduced mucin density in the old was accompanied by accumulation of trypsin and amylase in the intestinal wall, both when measured at the villi tips and at the level of the submucosa (FIG. 8). The oral trypsin inhibitor treatment partially restored the mucin layer in the old (FIG. 7) and attenuated the accumulation of these digestive enzymes in the intestinal wall (FIG. 8).

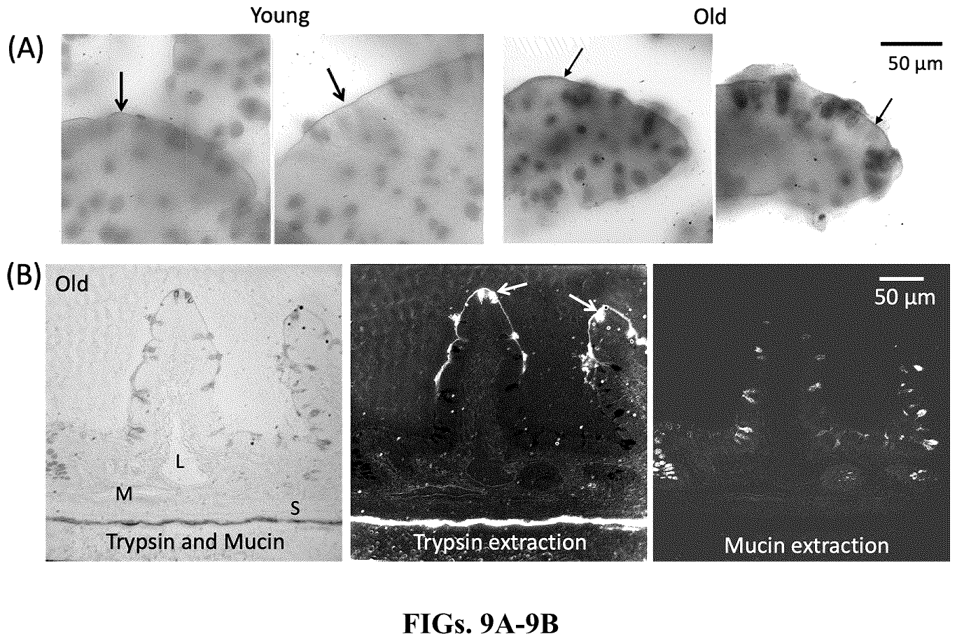

The loss of mucin was evident in the old includes goblet cell-associated mucin 13 (arrows) on the epithelial brush border and mucin 2 from goblet cells (FIG. 9A). Thin cross-sections of the intestinal villi in the old showed that the highest density of trypsin is at their tip, especially inside the residual cavities of goblet cells after mucin discharge, and in the epithelial brush border with reduced mucin label (FIG. 9B). Traces of trypsin label were detectable in the lamina propria, the microvasculature, lymphatics, and the intestinal serosa (see also FIGS. 1A-1F).

### Example 4: Collagen Degradation

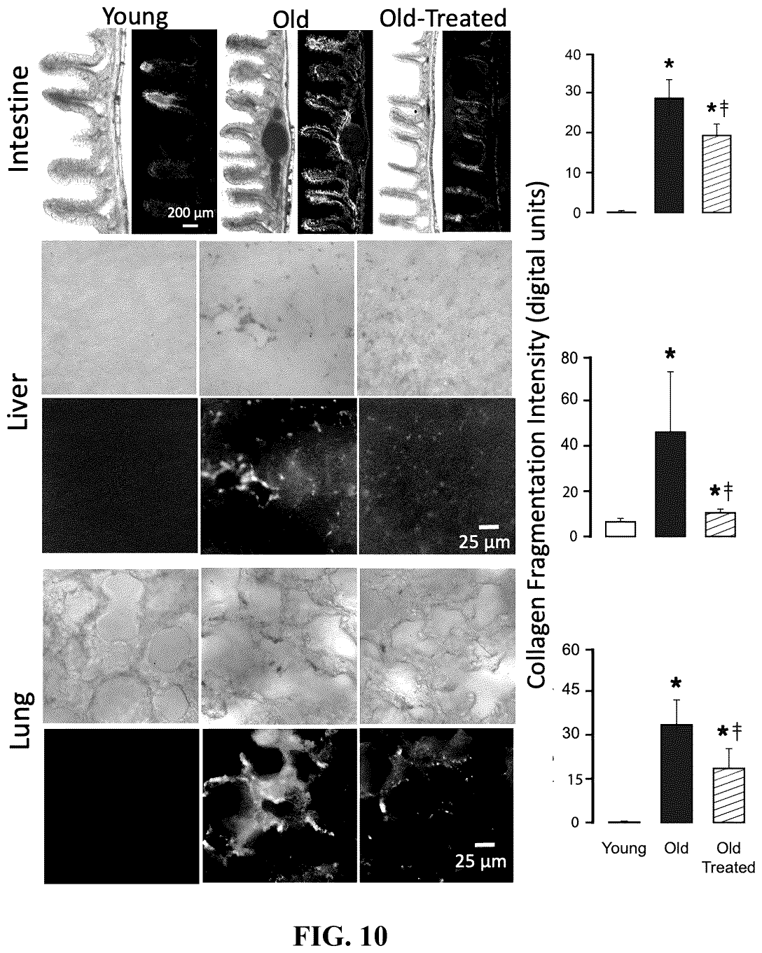

Organs of the young had low levels of collagen damage, detected by hybridizing peptides. In contrast, old organs had uniformly enhanced collagen damage in all organs that were studied (FIGS. 10-12). Collagen damage was significantly reduced by the two-week oral treatment with trypsin inhibitor.

Whereas in the intestine, collagen damage was present in the young and the old, and more villi in the old animals exhibited damage. The tips of the villi had the highest collagen damage in both young and old, which coincided with the location where digestive enzymes crossed the mucin epithelial barrier (FIG. 9B). The old had more villi and larger tissue areas in the lamina propria with collagen damage. The serosa also exhibited collagen damage in young and old at the same location where trypsin had accumulated (FIG. 9B).

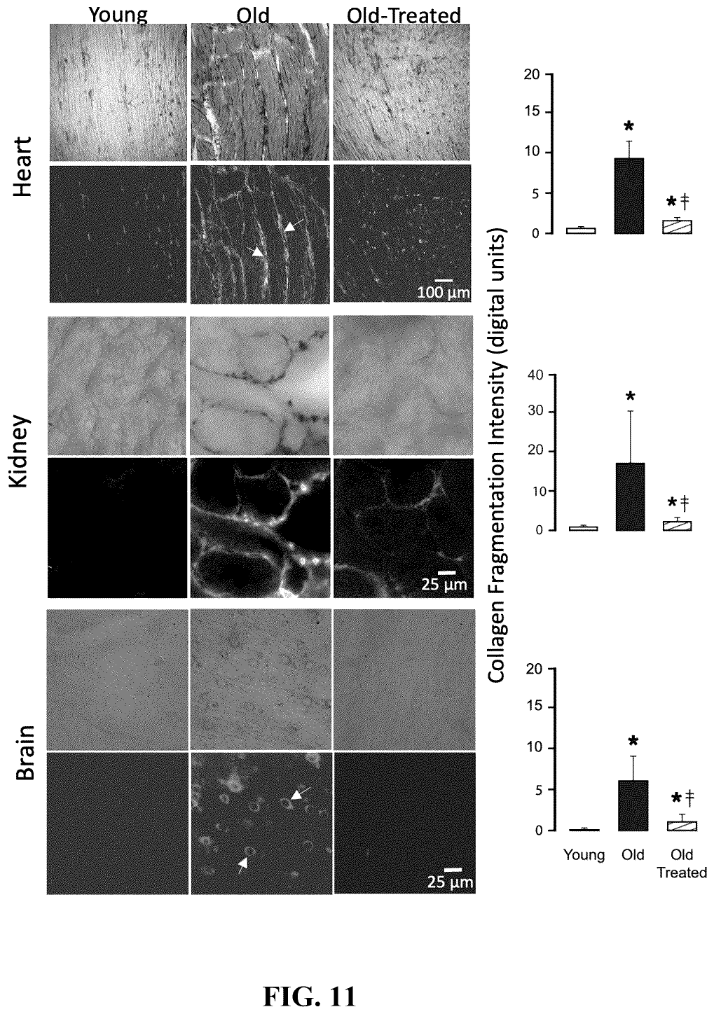

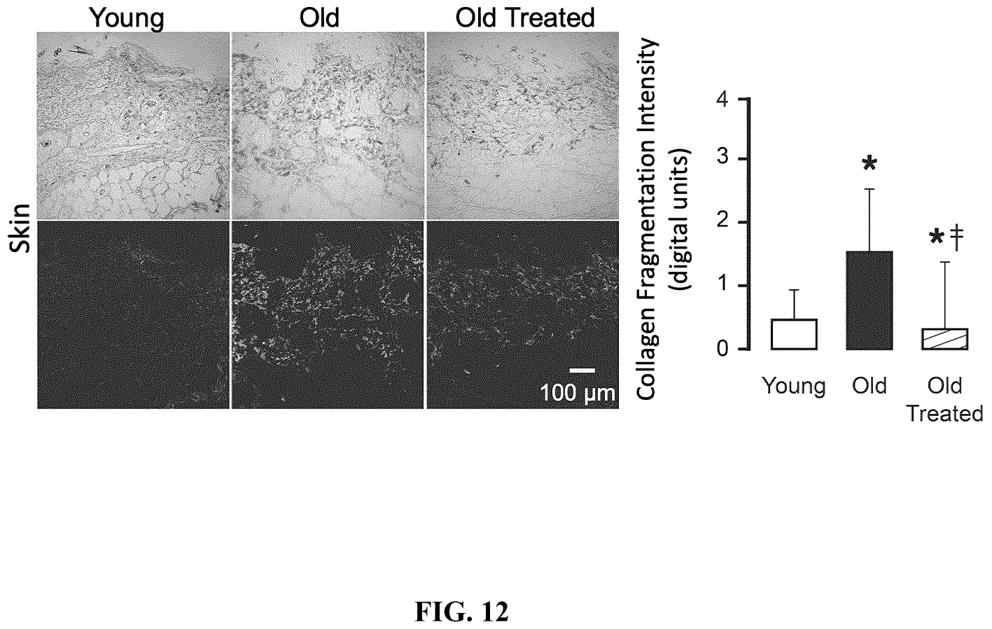

In the heart muscle, collagen degradation was accompanied by expansion of the interstitial space between muscle fibers (FIG. 11). In the skin, collagen damage occurred in the epidermis and dermis. In the brain, collage damaged was diffused throughout the tissue and enhanced in the wall of capillaries (FIG. 11). In the skin, collagen damage occurs in all collagen fibers of the epidermis and dermis of the old (FIG. 12). Just like in all other organs that were studied, collage degradation was reduced by oral blockade of pancreatic trypsin.

### Example 5: Insulin Receptor Cleavage

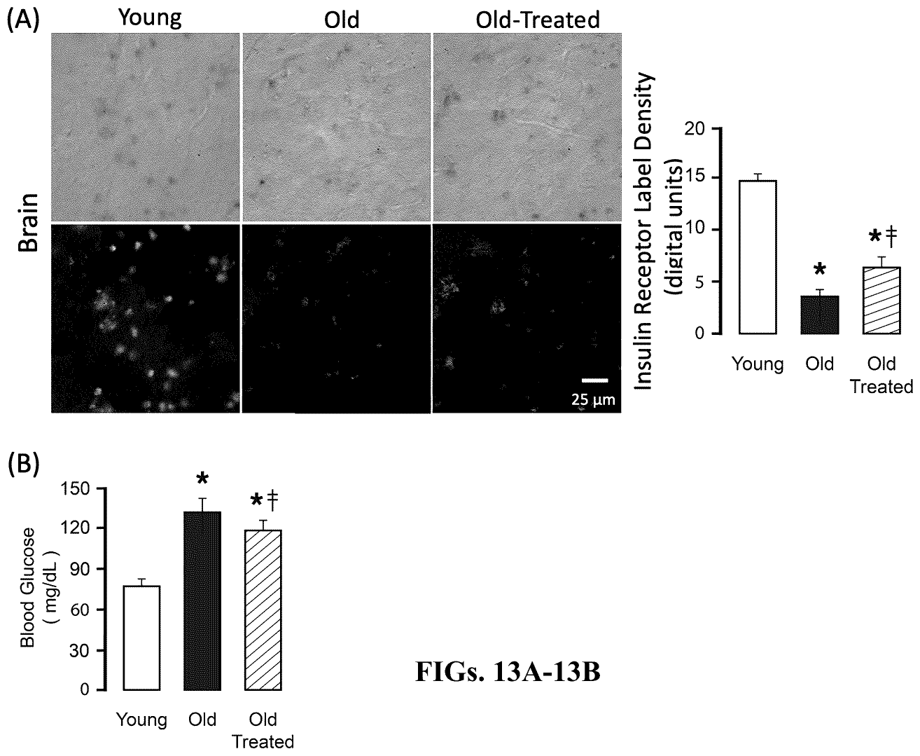

To determine whether digestive proteases may be involved in membrane receptor cleavage, the insulin receptor in the brain, an organ distant from the intestine, was investigated. Immunohistochemistry with a monoclonal antibody that binds to the extracellular domain of the insulin receptor showed its distribution in the cerebral cortex of the rat. The density of the insulin receptor ectodomains was significantly reduced in the aged, compared to the young, and in part restored after two-week trypsin inhibition (FIG. 13A). It coincided with an increase of plasma glucose levels in the old without, but not with the trypsin treatment (FIG. 13B). This reduction was accompanied by elevated plasma glucose levels in aged rats, an effect that was mitigated by two weeks of trypsin inhibition.

### Example 6: Progressive Multi-Tissue Degradation Mechanism Discovered

The current results in these rat studies brought to light a fundamental mechanism for progressive multi-tissue degradation in the aged that is a consequence of the need to digest. Whereas located in the lumen of the small intestine, multiple forms of pancreatic digestive enzymes were found in organs outside the lumen of the intestine of the old and less in the young. Pancreatic enzymes appeared even in the brain indicating that they breached two main barriers, the intestinal epithelial barrier and the blood-brain barrier. As demonstrated in the case of pancreatic trypsin, the enzymes escaped across the mucin-epithelial barrier in the small intestine and accumulated in vital organs. The digestive enzymes triggered a hallmark of aging, as detected by the breakdown of collagen, and they generated signatures for insulin resistance in form of hyperglycemia and extracellular insulin receptor cleavage. A two-week treatment of old rats with trypsin inhibitor restored in part the mucin-epithelial barrier, reduced the trypsin accumulation in vital organs, attenuated the collagen degradation, and restored in part the insulin receptor density and the blood glucose levels in the old.

### Digestive Enzyme Compartmentalization in the Gastrointestinal Tract

These results are in line with the central role of the gastrointestinal tract and the digestive enzymes in several diseases and multiorgan failure and death. A key requirement for the prevention of the degrading actions of the pancreatic digestive enzymes outside the gastrointestinal tract is their compartmentalization in the lumen of the pancreatic ducts and small intestine by the mucin/epithelial barrier. This barrier can be breached by multiple mechanisms, including, but not limited to, the reduction of the oxygen supply, the presence of partially digested food constituents, and unbound free fatty acids. Even in the young, the tip of the villi was infiltrated by digestive enzymes while also the site for epithelial cell apoptosis, suggesting that repeat injury and continuous growth of villi is part of a normal cycle during digestion. However, the current evidence suggests that chronic reconstitution of the intestinal villi is incomplete with reduced mucin density and enhanced digestive enzyme leak (FIGS. 7-9).

### Transport Pathways for Digestive Enzymes Out of the Intestine

Once digestive enzymes leaked across the epithelium/mucin barrier into the lamina propria of the intestinal villi, three pathways served to reach the systemic circulation. Digestive enzymes can be carried (a) via the intestinal microcirculation and the portal venous system, (b) via the intestinal and the mesenteric lymphatics and the lymphatic ducts into the venous circulation, bypassing the liver, and (c) across the submucosa, the muscularis, and the serosa of the intestine into the peritoneal fluid. The elevated label densities in the intestine and the liver of old rats for pancreatic lipase, elastase, and amylase, suggested a pathway via intestinal venules and hepatic portal veins. Other pathways involved in different stages of aging remain to be determined.

Within old organs, all tissue regions had an elevated digestive enzyme label density (FIGS. 1-6). The density was enhanced in the extracellular space (e.g. between heart muscle fibers; FIGS. 2C-2D), consistent with the fact that as water-soluble proteins without known membrane receptors, digestive enzymes have no effective transport mechanisms across intact cell membranes.

### Digestive Protease Activity in Aging

Pancreas digestive enzymes that escaped out of the small intestine were in an active form following conversion from their proform by enterokinases in the duodenum. Their activity in plasma and organs outside the intestine depended, however, on the levels of endogenous inhibitors (e.g. serpins synthesized in the liver) and serve to control digestive enzyme activity. However, endogenous inhibitors can be overwhelmed when larger amounts of digestive enzymes pass through the epithelial/mucin barrier into plasma, e.g. in an acutely ischemic intestine or during a postprandial period even in the young. The digestive enzyme activity, as a balance between enzyme and inhibitor concentrations, remains to be determined with in vivo zymographic techniques in aging.

### Tissue Degradation by Pancreatic Digestive Proteases

Digestive enzymes are optimized to degrade most biological tissues. Inside the lumen of the intestine, they are present in high concentrations, in an active state, and are relatively non-specific. Pancreatic trypsin, for example, degrades most proteins irrespective of the source and causes cell dysfunctions.

Once digestive proteases have breached the mucin/epithelial barrier, they in turn break down the mucin layer, cleave the extracellular domain of interepithelial junction proteins (E-cadherin), open the epithelial brush border, and even destroy the villi. Upon entry into organs outside the intestine, numerous cell and tissue functions are at risk by active digestive enzymes. Pancreatic trypsin in the circulation triggers the activation of pro-matrix metalloproteinases (proMMPs). The protease activity leads to ectodomain receptor cleavage and the reduction of their cell functions, such as cleavage of the insulin and leptin receptors with associated insulin and leptin resistance. The extent of surface receptor and glycocalyx cleavage in different organs of the old remains to be investigated and may constitute a mechanism for their spectrum of attenuated cell functions (e.g. protein homeostasis, nutrient sensing, stem cell exhaustion, intercellular communication) and chronic inflammation.

A key finding of the current study was the extensive cleavage of collagen in the organs we investigated (FIGS. 10-12). The breakdown of the collagen structure, detectable with hybridizing peptides binding to fractures in the triple-helical collagen molecule, can be produced either by mechanical stress or by exposure to proteases (e.g. trypsin) and precedes the collagen restructuring or loss of fibers. Collagen damage promotes the disassembly of integrin attachments, which in turn undermines integrin-dependent cell behavior, and enhances apoptosis. It was found that collagen damage mediated by pancreatic digestive proteases and the secondary enzymes they activate may thus be a central mechanism for biological aging.

The tissue-degrading processes by digestive enzymes observed here are in line with the coincidence of chronic diseases (e.g., diabetes) during aging and multi-organ failure at the end of life. The evidence disclosed herein supports the idea that a slow leak of digestive enzymes out of the gastrointestinal tract may lead to the gradual progression of organ dysfunction in aging, whereas a major breach of the mucin-epithelial barrier with a rapid escape of digestive enzymes leads to acute organ failure.

### Digestive Protease Inhibition

The current evidence from this study indicates that the accumulation of digestive enzymes in tissues outside the gastrointestinal tract in the old was reduced by two-week oral trypsin inhibition. This intervention needs to be nuanced to block autodigestion but not digestion. Even though the trypsin inhibitor was administered orally, the concentration in the drinking water was kept sufficiently low so that a temporary treatment did not lead to a detectable attenuation of digestion, such as a reduction of body weight. The strategy served to restore the mucin layer on the intestinal villi (FIG. 7), reduce the leak of digestive enzymes into the intestinal wall (FIGS. 1, 4, and 8), and accumulation of digestive enzymes in organs outside the intestine (FIGS. 1-6).

### Aging Interventions and Autodigestion

In multiple species, caloric reduction without starvation or timed-eating attenuates age-associated morbidities. The autodigestion hypothesis disclosed herein may provide an insight for such a benefit. A single meal can be accompanied by an instantaneous leakage of digestive enzymes into the central circulation within less than an hour of food consumption. Reduction in the daily frequency and the volume of food passing through the small intestine may attenuate damage to the mucin/epithelial barrier and consequently reduce enzyme leak. In light of the continuous repair of the intestinal epithelium, prolonging the periods between meals may enhance the reconstitution of the microvilli and the epithelial/mucin barrier and thereby minimize autodigestion. The exact chronology of intestinal damage by leaking digestive enzymes and repair of the villi with their mucin/epithelial barrier during and after a meal remains to be elucidated.

### Example 7: Exemplary Treatment of Potential Conditions in Patients-Mild Cognitive Impairment

A patient presents at the doctor with forgetfulness and memory problems. The doctor assesses the patient and determines that the patient likely has mild cognitive impairment. The doctor prescribes chronic administration of a pharmaceutical composition disclosed herein for the treatment of the mild cognitive impairment. The patient's mild cognitive impairment symptoms stabilize.

### Example 8: Exemplary Treatment of Potential Conditions in Patients-Kidney

A patient presents at the doctor with decreased urine output and fatigue. The doctor assesses the patient and determines that the patient likely has symptoms of kidney failure. The doctor prescribes chronic administration of a pharmaceutical composition disclosed herein for the treatment of the kidney failure symptoms. The patient's kidney failure symptoms improve.

### Example 9: Exemplary Treatment of Potential Conditions in Patients-Liver

A patient presents at the doctor with jaundice and nausea. The doctor assesses the patient and determines that the patient likely has symptoms of liver failure. The doctor prescribes chronic administration of a pharmaceutical composition disclosed herein for the treatment of the liver failure symptoms. The patient's liver failure symptoms stabilize.

### Example 10: Exemplary Treatment of Potential Conditions in Patients-Lung

A patient presents at the doctor with shortness of breath and cough. The doctor assesses the patient and determines that the patient likely has symptoms of lung disease. The doctor prescribes chronic administration of a pharmaceutical composition disclosed herein for the treatment of the lung disease symptoms. The patient's lung disease symptoms stabilize.

### Example 11: Exemplary Treatment of Potential Conditions in Patients-Intestine

A patient presents at the doctor with dyspepsia, bloating, and nausea. The doctor assesses the patient and determines that the patient likely has symptoms of intestinal perforation/leaky gut. The doctor prescribes chronic administration of a pharmaceutical composition disclosed herein for the treatment of the leaky gut symptoms. The patient's leaky gut symptoms stabilize.

### Example 12: Exemplary Treatment of Potential Conditions in Patients-Tremor

A patient presents at the doctor with shaking in the hands and difficulty writing or drawing. The doctor assesses the patient and determines that the patient likely has symptoms of tremor. The doctor prescribes chronic administration of a pharmaceutical composition disclosed herein for the treatment of the patient's tremor symptoms. One or more of the patient's tremor symptoms generally stabilize.

### Example 13: Exemplary Treatment of Potential Conditions in Patients-Unstable Blood Pressure

A patient presents at the doctor with unstable blood pressure. The doctor assesses the patient and determines that the patient has unstable blood pressure in need of treatment. The doctor prescribes chronic administration of a pharmaceutical composition disclosed herein for the treatment of the patient's unstable blood pressure. The patient's blood pressure stabilizes.

### Example 14: Exemplary Treatment of General Biological Aging in Older Patients

An older patient presents at the doctor with mild loss of muscle strength, mild loss of sight and hearing, and mild loss of energy compared to a few years ago. The doctor assesses the patient and determines that the patient does not currently meet the criteria for any particular disease or condition, and that these symptoms are likely due to biological aging. The doctor prescribes chronic administration of a pharmaceutical composition disclosed herein for the treatment of the patient's symptoms. One or more of the patient's symptoms generally stabilize.

### Example 15: Exemplary Treatment of Potential Conditions in Patients-Dementia

A patient presents at the doctor with short-term memory loss, Difficulty finding the right words, and changes in mood or behavior. The doctor assesses the patient and determines that the patient likely has a risk of developing dementia. The doctor prescribes chronic administration of a pharmaceutical composition disclosed herein for the treatment and/or prevention of the dementia. The patient's early symptoms of dementia stabilize and do not worsen.

### Example 16: Exemplary Treatment of Potential Conditions in Patients-Type 2 Diabetes

A patient presents at the doctor with polydipsia, polyuria, and fatigue. The doctor assesses the patient and determines that the patient has type 2 diabetes. The doctor prescribes chronic administration of a pharmaceutical composition disclosed herein for the treatment of the type 2 diabetes. The patient's symptoms of type 2 diabetes stabilize.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1.-63. (canceled)

64. A method of reversing accumulation of a lipase in an organ of a subject, comprising:
selecting a subject having or at risk of accumulation of a lipase in the organ; and
administering a therapeutically effective amount of a lipase inhibitor to the subject, thereby reversing accumulation of the lipase in the organ of the subject.

65. The method of claim 64, wherein the subject is at least 40 years old, at least 50 years old, or at least 60 years old, and wherein the subject is not at risk of developing shock and/or septic shock, and wherein the subject does not have HIV.

66. The method of claim 64, wherein the organ is one or more of a small intestine, liver, lung, heart, kidney, brain, or skin.

67. The method of claim 66, wherein the subject has: a brain disease or condition that is one or more of mild cognitive impairment, Alzheimer's Disease, dementias including frontotemporal dementia, epilepsy or other seizure disorders, mental disorder, multiple sclerosis, Huntington's Disease, Parkinson's Disease, amyotrophic lateral sclerosis, meningitis, encephalitis, brain cancer, Crutzfeldt-Jakob disease, chronic traumatic encephalopathy, long-haul COVID-associated dementia, or stroke; a heart disease or condition that is one or more of coronary heart disease, angina, unstable angina, heart failure, cardiac arrhythmias, valve disease, high blood pressure, heart arrhythmias, endocarditis, pericardial disease, or cardiomyopathy; a kidney disease or condition that is one or more of chronic kidney disease, diabetic kidney disease, acute kidney injury, kidney stones, kidney infections, including pyelonephritis, kidney cysts, or kidney cancer; or a liver disease or condition that is one or more of hepatitis A, hepatitis B, hepatitis C, autoimmune hepatitis, primary biliary cholangitis, primary sclerosing cholangitis, hemochromatosis, Wilson's disease, alpha-1 antitrypsin deficiency, liver cancer, bile duct cancer, liver adenoma, nonalcoholic fatty liver disease, or nonalcoholic steatohepatitis.

68. The method of claim 64, wherein the lipase inhibitor is a competitive inhibitor.

69. The method of claim 68, wherein the lipase inhibitor is one or more of tetrahydrolipstatin (orlistat), cetilistat, valilactone, percyquinin, panclicins A-E, ebelactone A and B, vibralactone, esterastin, nafamostat mesylate (FUT-175), and lipstatin.

70. The method of claim 69, wherein the therapeutically effective amount of the lipase inhibitor is less than about 10% of a digestive enzyme activity of the subject.

71. The method of claim 64, wherein the therapeutically effective amount of the lipase inhibitor is less than about 10 μM.

72. The method of claim 64, wherein the therapeutically effective amount of the lipase inhibitor is less than about 5 μM.

73. The method of claim 64, wherein the lipase inhibitor is enterally administered, intraperitoneally administered, intravenously administered, intramuscularly administered, subcutaneously administered, intracutaneously administered, orally administered, intranasally administered, intrapulmonarily administered, intrarectally administered, or administered by a telemetry-controlled external or implanted infusion pump that is directed toward the organ, or wherein the lipase inhibitor is administered as an eye drop, and wherein the lipase inhibitor is administered for more than about one week, more than about two weeks, or more than about four weeks.

74. The method of claim 64, wherein the lipase inhibitor is administered as a liposome composition or as a nanoparticle encapsulation.

75. The method of claim 64, wherein reversing accumulation of the lipase in the organ of the subject reverses cellular damage in the organ of a subject.

76. A method of treating type 2 diabetes in a subject, comprising:
administering a therapeutically effective amount of a lipase inhibitor to the subject, thereby reducing a cleavage of one or more insulin receptors by a lipase in an organ of the subject.

77. A pharmaceutical composition for the treatment of aging or age-related conditions in a subject, the pharmaceutical composition comprising a lipase inhibitor.

78. The pharmaceutical composition of claim 77, wherein the age-related conditions affect an organ of one or more of a brain, spinal cord, heart, kidney, muscle, liver, or lung, and wherein the age-related conditions comprise one or more of mild cognitive impairment, Alzheimer's Disease, dementias including frontotemporal dementia, age-related loss of neuronal function, including but not limited to memory, balance, sensation, pain, epilepsy or other seizure disorders, mental disorder, multiple sclerosis, Huntington's Disease, Parkinson's Disease, amyotrophic lateral sclerosis meningitis, encephalitis, brain cancer, or transient ischemic strokes; one or more of coronary heart disease, angina, unstable angina, heart failure, valve disease high blood pressure, heart arrhythmias, endocarditis, pericardial disease, and cardiomyopathy; one or more of fibromyalgia, myositis, including polymyositis and dermatomyositis, muscular dystrophy, myasthenia gravis, amyotrophic lateral sclerosis, rhabdomyolysis, cardiomyopathy, sarcopenia, Charcot-Marie-Tooth disease, multiple sclerosis, myopathy, peripheral neuropathy, or spinal muscular atrophy; one or more of acute kidney injury, kidney stones, kidney infections, including pyelonephritis, kidney cysts, the subject being in need of renal dialysis, or kidney cancer; or one or more of hepatitis A, hepatitis B, hepatitis C, autoimmune hepatitis, primary biliary cholangitis, primary sclerosing cholangitis, hemochromatosis, Wilson's disease, alpha-1 antitrypsin deficiency, liver cancer, bile duct cancer, liver adenoma, nonalcoholic fatty liver disease, or nonalcoholic steatohepatitis.

79. The pharmaceutical composition of claim 77, wherein the lipase inhibitor is a competitive inhibitor, and wherein the lipase inhibitor is one or more of tetrahydrolipstatin (orlistat), cetilistat, valilactone, percyquinin, panclicins A-E, ebelactone A and B, vibralactone, esterastin, nafamostat mesylate (FUT-175), and lipstatin.

80. The pharmaceutical composition of claim 79, wherein the lipase inhibitor is administered at less than 10% of a digestive enzyme activity of the subject.

81. The pharmaceutical composition of claim 80, wherein the lipase inhibitor is less than 10 μM or is less than 5 μM.

82. The pharmaceutical composition of claim 77, wherein the lipase inhibitor is enterally administered, intraperitoneally administered, intravenously administered, intramuscularly administered, subcutaneously administered, intracutaneously administered, orally administered, intranasally administered, intrapulmonarily administered, intrarectally administered, or administered by a telemetry-controlled external or implanted infusion pump, or wherein the lipase inhibitor is administered by a telemetry-controlled infusion pump, or wherein the lipase inhibitor is administered as a liposome composition or a nanoparticle directed toward the organ of the subject, or wherein the lipase inhibitor is administered for more than one week, more than two weeks, or more than four weeks.

83. A method of treating dementia in a subject, comprising: administering a therapeutically effective amount of the pharmaceutical composition of claim 78 to the subject, thereby reducing or reversing an accumulation of lipase in a brain of the subject.
